(19) United States
(12) Patent Application Publication
(10) Pub. No.: US 2009/0317330 A2
(43) Pub. Date: Dec. 24, 2009

(54) DIAGNOSIS AND TREATMENT OF DISEASES INVOLVING PLATELET ACTIVATION

(75) Inventor: Karlheinz Peter, Hawthorn East Victoria (AU)

Correspondence Address: CLARK & ELBING LLP, 101 FEDERAL STREET, BOSTON, MA, 02110, UNITED STATES, 617-428-0200, 617-428-7045, patentadministrator@clarkelbing.com

(73) Assignee: Baker IDI Heart and Diabetes Institute Holdings Limited, Melbourne Victoria (AU)

(21) Appl. No.: 12/317,464

(22) Filed: Dec. 22, 2008

Related U.S. Application Data
(60) Provisional application No. 61/008,990, filed on Dec. 21, 2007.

(51) Int. Cl. A61K 49/00

(52) U.S. Cl. 424/9.1

(57) ABSTRACT

Disclosed herein are compositions, methods, and kits for diagnosing or predicting a disease in a subject, for detecting activated platelets in a subject, and for treating a disease in a subject, wherein the diseases are mediated by platelet activation.

### CROSS-REFERENCE TO RELATED APPLICATIONS

This application claims the benefit of the filing date of U.S. Provisional Application Ser. No. 61/008,990, filed Dec. 21, 2007, which is hereby incorporated by reference.

### TECHNICAL FIELD

The present invention relates to compositions, methods and kits for diagnosing or predicting a disease in a subject, for detecting activated platelets in a subject and for treating a disease in a subject, wherein said diseases are mediated by platelet activation.

### BACKGROUND OF THE INVENTION

Diseases involving platelet activation include stroke, thrombosis, cardiovascular disease, inflammatory diseases, autoimmune diseases, immunoinflammatory diseases, allergic diseases, predispositions thereto, infectious diseases and/or cancer. In one such disease, cerebral malaria (CM), platelet sequestration in the cerebral microvasculature plays a pivotal role in the pathogenesis of CM. The histopathology of mice infected with Plasmodium berghei ANKA reveals extensive damage to vascular endothelial cells and plugging of vessels caused by platelet thrombi. Similarly, immunohistochemistry for the platelet-specific glycoprotein IIb/IIIa-receptor (GPIIb/IIIa), the activated conformation of which is responsible for platelet linkage via fibrinogen, reveals that platelet accumulation occurs in the microvasculature of patients with CM.

The mechanisms of platelet activation, aggregation and adhesion are not understood. However, the local production of various cytokines may be a contributing factor. Cytokines are involved in the recruitment of distinct populations of leukocytes across the intact brain endothelium despite the induction of the same pattern of adhesion molecule expression. The differential induction of chemokines could determine which populations are recruited, but it is not known whether platelet adhesion to the brain microvasculature is dependent on the expression of specific cytokines.

A non-invasive approach for the specific detection of activated platelets or platelet thrombi in the cerebral microvasculature under different conditions of cytokine expression could help in determining the influence of cytokines on vascular platelet adhesion in a broad range of diseases involving platelet activation. Recent progress in MRI techniques has enabled the detection of some molecular targets by designing contrast agents that will bind to cellular receptors or surface antigens. By delivering high payloads of contrast agent such as iron oxide particles to molecular epitopes, imaging of even sparsely distributed molecules may be possible.

### SUMMARY OF THE INVENTION

According to a first aspect of the present invention there is provided a method for diagnosing or predicting a disease in a subject, wherein the method comprises administering to the subject a compound comprising:

(a) a binding element capable of specifically binding to an activated platelet; and

(b) an imaging agent

wherein binding of the compound to an activated platelet is indicative of the disease.

According to a second aspect of the present invention, there is provided a method for detecting aggregation of platelets in a subject, wherein the method comprises administering to the subject a compound comprising:

(a) a binding element capable of specifically binding to an activated platelet; and

(b) an imaging agent

wherein binding of the compound to an activated platelet is indicative of the disease.

The methods may comprise an immunoassay. The immunoassay may comprise magnetic resonance imaging.

The methods may further comprise determining a level of cellular expression of at least one additional molecule.

The at least one additional molecule may be a polynucleotide or a polypeptide encoded thereby.

The polynucleotide may encode tumour necrosis factor (TNF).

The polypeptide may comprise tumour necrosis factor (TNF).

The polypeptide may be intracellular, cell surface-associated or secreted.

The methods may be used for diagnosing, predicting or monitoring stroke, thrombosis, cardiovascular disease, inflammatory diseases, autoimmune diseases, immunoinflammatory diseases, allergic diseases, predispositions thereto, infectious diseases, cancer and/or cancer treatment.

The methods may be used for predicting or monitoring responses to therapy for stroke, thrombosis, cardiovascular disease, inflammatory diseases, autoimmune diseases, immunoinflammatory diseases, allergic diseases, predispositions thereto, infectious diseases and/or cancer.

The methods may be used for the early diagnosis of diseases, particularly in, but not limited to, situations where the disease is not yet manifested in clinically detectable symptoms, or is otherwise not detectable by other methods.

According to a third aspect of the present invention, there is provided a method for treating a disease in a subject, wherein said method comprises administering to the subject a compound comprising:

(a) a binding element capable of specifically binding to an activated platelet; and

(b) an agent that inhibits TNF

wherein binding of the compound to an activated platelet facilitates inhibition of TNF signaling by the agent.

According to a fourth aspect, the invention provides a method of non-invasively detecting vascular platelet aggregation in a subject comprising:

(a) administering to the subject a composition comprising a binding element that specifically binds activated platelets conjugated to an imaging agent, wherein the composition has substantially no effect upon platelet aggregation;

(b) allowing the binding element to bind to any activated platelets present in the subject; and

(c) imaging the imaging agent, wherein an image signals the detection of vascular platelet aggregation.

According to a fifth aspect, the invention provides methods further comprising the steps of:

(d) allowing for clearance of the imaging agent from the subject sufficient to eliminate or reduce its detection; and

(e) repeating steps (a) through (c).

In a sixth aspect, the steps (d) and (e) are repeated at least once, and may be repeated 2, 3, 4, 5, 6, 7, 8, 9, 10, 11, 12, 13, 14, 15, 16, 17, 18, 19, 20, 30, 40, 50, or 100 times, or more.

In additional aspects, the methods of the invention include a method of monitoring a therapy wherein imaging of the imaging agent indicates the success or failure of the therapy. The therapies contemplated for monitoring include all therapies directed to treatment of pathologies characterized by the aggregation of platelets. Indeed, any condition or therapy involving the aggregation of platelets is within the scope of the methods of the invention. In particular instances, the therapies may include therapies directed to treatment of stroke, thrombosis, cardiovascular disease, inflammatory diseases, autoimmune diseases, immunoinflammatory diseases, allergic diseases, predispositions thereto, infectious diseases or cancer.

The methods and compositions of the invention may be adapted to or applied to use in any animal that comprises platelet cells, including vertebrates. Especially contemplated subjects include mammals, and particularly humans.

In further aspects of the invention, the imaging agent comprises a contrast agent for magnetic resonance imaging. In a related embodiment, the imaging comprises magnetic resonance imaging.

In additional aspects, the binding element is an antibody. In a preferred aspect, the binding element is a single chain antibody.

In additional aspects, the binding element is less than 34 kDa in size.

In another aspect, the binding element conjugated to the imaging agent is a complex less than 45 kDa, 55 kDa, 65 kDa, 75 kDa, 85 kDa, 95 kDa, or 100 kDa in size.

### DEFINITIONS

Throughout this specification and the claims, unless the context requires otherwise, the word “comprise” and its variations, such as “comprises” and “comprising”, will be understood to imply the inclusion of a stated integer or step or group of integers but not the exclusion of any other integer or step or group of integers or steps.

The term “expression” as used herein refers interchangeably to expression of a gene or gene product, including the encoded polypeptide or protein. Expression of a gene product may be determined, for example, by immunoassay using an antibody(ies) that bind with the polypeptide. Alternatively, expression of a gene may be determined by, for example, measurement of mRNA (messenger RNA) levels.

As used herein the terms “polynucleotide” and “nucleic acid” are used interchangeably. The term “polynucleotide” refers to a single- or double-stranded polymer of deoxyribonucleotide or ribonucleotide bases, or fragments, analogues, derivatives, or combinations thereof. The terms include reference to any specified sequences as well as to the sequences complementary thereto, unless otherwise indicated. It will be understood that “5′end” as used herein in relation to a nucleic acid molecule corresponds to the N-terminus of the encoded polypeptide and “3′end” corresponds to the C-terminus of the encoded polypeptide.

As used herein the term “oligonucleotide” means a single-stranded nucleic acid capable of acting as a point of initiation of template-directed nucleic acid synthesis. An oligonucleotide is a single-stranded nucleic acid typically ranging in length from 2 to about 500 bases. The precise length of an oligonucleotide will vary according to the particular application, but typically ranges from 15 to 30 nucleotides. An oligonucleotide need not reflect the exact sequence of the template but must be sufficiently complimentary to hybridize to the template, thereby facilitating preferential amplification of a target sequence. Thus, a reference to an oligonucleotide as being “specific” for a particular gene or gene product, such as mRNA, includes within its scope an oligonucleotide that comprises a complementarity of sequence sufficient to preferentially hybridize to the template, without necessarily reflecting the exact sequence of the target polynucleotide.

The term “analogue” when used in relation to a polynucleotide or residue thereof, means a compound having a physical structure that is related to a DNA or RNA molecule or residue, and preferably is capable of forming a hydrogen bond with a DNA or RNA residue or an analogue thereof (i.e., it is able to anneal with a DNA or RNA residue or an analogue thereof to form a base-pair). Such analogues may possess different chemical and biological properties to the ribonucleotide or deoxyribonucleotide residue to which they are structurally related. Methylated, iodinated, brominated or biotinylated residues are examples of analogues.

The term “derivative” when used in relation to a polynucleotide of the present invention includes any functionally-equivalent nucleic acids, including any fusion molecules produced integrally (e.g., by recombinant means) or added post-synthesis (e.g., by chemical means). Such fusions may comprise one or both strands of the double-stranded oligonucleotide of the invention with RNA or DNA added thereto or conjugated to a polypeptide (e.g., puromycin or other polypeptide), a small molecule (e.g., psoralen) or an antibody.

As used herein the terms “polypeptide”, “peptide” and “protein” are used interchangeably. The term “polypeptide” means any polymer made up of amino acids linked together by peptide bonds. Accordingly, the term “polypeptide” includes within its scope a full length protein and fragments thereof, together with analogues and variants thereof.

The term “fragment” when used in relation to a polypeptide or polynucleotide molecule refers to a constituent of a polypeptide or polynucleotide. Typically the fragment possesses qualitative biological activity in common with the polypeptide or polynucleotide. A polypeptide fragment may be between about 5 to about 150 amino acids in length, between about 5 to about 100 amino acids in length, between about 5 to about 50 amino acids in length, or between about 5 to about 25 amino acids in length. Alternatively, the peptide fragment may be between about 5 to about 15 amino acids in length. However, fragments of a polynucleotide do not necessarily need to encode polypeptides which retain biological activity. Rather, a fragment may, for example, be useful as a hybridization probe or PCR oligonucleotide. The fragment may be derived from a polynucleotide of the invention or alternatively may be synthesized by some other means, for example chemical synthesis.

The term “analogue” as used herein with reference to a polypeptide means a polypeptide which is a derivative of the polypeptide of the invention, which derivative comprises addition, deletion or substitution of one or more amino acids, such that the polypeptide retains substantially the same function.

The term “variant” as used herein refers to substantially similar sequences. Generally, polypeptide or polynucleotide sequence variants possess qualitative biological activity in common. Further, these polypeptide or polynucleotide sequence variants may share at least 50%, 55%, 60%, 65%, 70%, 75%, 80%, 85%, 90%, 95%, 96%, 97%, 98% or 99% sequence identity. Also included within the meaning of the term “variant” are homologues of polypeptides or polynucleotides of the invention. A homologue is typically a polypeptide or polynucleotide from a different species but sharing substantially the same biological function or activity as the corresponding polypeptide or polynucleotide disclosed herein.

As used herein the terms “treating” and “treatment” refer to any and all uses which remedy a condition or symptoms, prevent the establishment of a condition or disease, or otherwise prevent, hinder, retard, ameliorate or reverse the progression of a condition or disease or other undesirable symptoms in any way whatsoever.

As used herein the term “effective amount” includes within its meaning a non-toxic but sufficient amount of an agent or compound to provide the desired effect. The exact amount required will vary from subject to subject depending on factors such as the species being treated, the age and general condition of the subject, the particular agent being administered and the mode of administration and so forth. Thus, it is not possible to specify an exact “effective amount”. However, for any given case, an appropriate “effective amount” may be determined by one of ordinary skill in the art using only routine experimentation.

As used herein, the term “antibody” includes antibody fragments, including but not limited to, heavy chains, light chains, variable regions, constant regions, Fab, Fc, Fc receptors, single chain (scFv) antibodies, complementarity determining regions (CDRs) and any protein, polypeptide or peptide comprising an antibody, or part thereof.

As used herein the term “substantially” means the majority but not necessarily all.

The reference to any prior art in this specification is not, and should not be taken as an acknowledgement or any form of suggestion that prior art forms part of the common general knowledge.

Abbreviations used herein include:

ADP adenosine diphosphate

BBB blood brain barrier

CM cerebral malaria

CNS central nervous system

rCVB regional cerebral blood volume

ECG electrocardiogram

Gd-DTPA Gadopentetic acid

GLUT 1 glucose transporter 1

GPIIb/IIIa glycoprotein IIb/IIIa

HIV human immunodeficiency virus

ICAM-1 intercellular adhesion molecule 1

IL-1□ interleukin-1□

LFA-1 lymphocyte function-associated antigen-1

LIBS ligand induced binding sites

LIBS-MP1O a single-chain antibody conjugated to microparticles of iron oxide

LT-□ lymphotoxin-□

mAB monoclonal antibody

MPIO microparticles of iron oxide

MRI magnetic Resonance Imaging

PCR polymerase chain reaction

ppm parts per million

scFv single chain antibody

TNF tumor necrosis factor

VCAM-1 vascular cellular adhesion molecule 1

### BRIEF DESCRIPTION OF THE DRAWINGS

The application file contains drawings executed in color (FIGS. 1-9). Copies of this patent or patent application with color drawings will be provided by the Office upon request and payment of the necessary fee,

Embodiments of the present invention will now be described, by way of example only, with reference to the accompanying drawings:

FIGS. 1A-1D: Conventional imaging of murine cerebral malaria. (A) Coronal T₁-weighted image acquired 7 days after the injection of 10⁶ P. berghei ANKA-pRBC. (B) T₁-weighted image from the same animal following injection of Gd-DTPA. Note the regions of increased signal intensity compared to (A), indicating blood-brain barrier breakdown (arrow). (C) T₂-weighted image of the same slice showing increased signal intensity in the same regions as the Gd-DTPA enhancement. (D) Graph showing a significant (p<0.05) elevation in T₂ in the hippocampus of day 7 terminal-stage CM mice.

FIGS. 2A-2C: In vitro MPIO platelet binding. Non-activated (left column) or ADP-activated (right column) platelets were incubated with LIBS-MPIO (A), with control-MPIO (B) or non-functionalized MPIOs (C). Note the presence of LIBS-MPIO binding on the surface of the activated platelets on the thrombus in (A), which is not observed on non-activated platelets or with the control MPIO or non-functionalized MPIO (B & C).

FIGS. 3A-3E: Data from animals with cerebral malaria and LIBS-MPIO contrast agent injection. T₂*-weighted 3D gradient-echo images from CM-mice following intravenous injection of LIBS-MPIO (A) and control-MPIO (B) are presented in two representative slices at two different levels within the same brain. Areas of MPIO-induced signal appear as dark signal voids in cortical regions of the LIBS-MPIO (arrows), but not in the control-MPIO injected animal. 3D reconstruction confirms the cortical binding pattern in LIBS-MPIO injected mice (C), whereas only modest background binding is evident in the control-MPIO animal (D); quantification of signal voids demonstrated a significant difference between the LIBS-MPIO and control-MPIO injected animals (E).

FIGS. 4A-4D: In vivo T₂*-weighted coronal images (in rows of 4 images per brain, beginning at bregma and moving backwards in 700 μm increments) from 3D gradient-echo data sets each with ˜90 μm isotropic resolution. (A) Animal injected intrastriatally with 1 μg TNF in 0.5 μl saline 11.5 h prior to intravenous injection of LIBS-MPIO (˜4.5 mg/kg Fe). Intense low-signal areas (i.e. black) reflect the specific retention of MPIO on activated platelets adhering to the cerebrovascular endothelium. In contrast, no effects of the LIBS-MPIO agent were detected in animals injected 11.5 h previously with either 1 μg IL-1β in 0.5 μl saline (B) or 0.50 □l saline alone (C). Similarly, no non-specific effects of the control-MPIO contrast agent could be detected in animals injected intrastriatally with 1 μg TNF in 0.5 μl saline (D).

FIGS. 5A-5C: LIBS-MPIO binding pattern after intracerebral TNF-injection in a 3D-reconstruction (A), showing the enhancement of cortical and central LIBS-MPIO binding. Minimal background binding is observed in animals with control-MPIO injection (B). Quantitative data evaluation shows a significantly higher binding of LIBS-MPIO to TNF-injected brain areas compared to all controls (C).

FIGS. 6A-6E: Histology of TNF-injected brains with LIBS-MPIO injection. Cresyl-violet stain reveals binding of MPIO to areas on the vascular wall as highlighted by arrows (A). Binding of LIBS-MPIO to platelets or platelet thrombus is confirmed using immunohistochemistry for platelet-specific CD41 (B): two beads appearing in different focus and therefore of different shape can be recognized at areas of platelet aggregation (arrows). Similarly, MPIOs can be detected on platelets and platelet thrombi in animals with CM (arrows, C). (D) Simultaneous depiction of platelet positive elements per injected hemisphere at 6, 12 and 24 hours after intracerebral injection of TNF (diamonds) or saline (squares) on the left hand ordinate in mice, and quantification of LIBS-MPIO-induced signal void in animals 6, 12 and 24 h after intracerebral TNF-injection on the right hand ordinate (E). There is a rough correlation between number of platelet positive elements over time and the LIBS-MPIO-induced signal void.

FIGS. 7A-7F: Platelet and leukocyte accumulation in the brain following the intrastriatal injection of TNF. (Ai) Platelets localised within blood vessel in the brain close to the injection site [brown label (DAB) as represented by the lighter shaded area—see Arrow-‘1’ as example] with a cresyl-violet counter stain as represented by the darker shaded objects—see Arrow-‘2’ as example. (Aii) Confocal triple-labelling immunocytochemistry identifies platelets (Arrow-‘3’ as example—red) and ED-1 positive cells (Arrow-‘4’ as example—green) inside a vessel with an intact endothelium as revealed by GLUT1 (Arrow-‘5’ as example—blue) staining. Scale bar represents 20 μm. (B) The number of GPIIa/IIIb positive elements in the injected hemisphere following the injection of 1 μg of TNF (diamonds), or 1 ng IL-1 (triangles), or saline (squares) in a volume of 1 μl for rats or 0.5 μl for mice was microinjected into the striatum. (C) Total brain leukocytes as identified by leukocyte common antigen, (D) ED-1-positive recruited monocytes & activated microglial cells present in the brain parenchyma and (E) ED-1-positive recruited monocytes associated with the lumenal portion of the brain vasculature detected by immunohistochemistry over 24 h following intrastriatal injection of TNF (squares) or saline vehicle (diamonds) are presented. Representative photographs showing the absence of leukocytes in the meninges (F i) or parenchyma (F ii) and their presence (Arrow-‘6’ as example) in the parenchyma following injection of TNF (F iii) are presented after immunohistochemical labelling for leukocyte common antigen. Double-labelling immunocytochemistry (F iv, F vi) and confocal imaging (F v, F vii) of ED-1-positive cells and the brain vasculature (as identified by GLUT-1 Arrow-‘a’) highlight the differences in mononuclear cell recruitment patterns in the parenchyma (F iv, F v) and meninges (F vi, F vii). Note in the meninges the presence of large numbers of ED-1 [blue (VIP): Arrow-‘a’ ] positive cells that appear to have free passage from the vasculature [brown (DAB): Arrow-‘b’)] (scale bar=10 μm) in comparison to the same cells in the parenchyma that appear vessel associated (inset in F vi: high power of extravascular ED-1 positive cells: Arrow-‘a’). ELISA results are expressed as pg MCP-1 per mg of total protein ±standard error of mean. Cell numbers in brain are expressed per mm²±standard error of mean. Asterisk denotes p<0.05 compared to saline vehicle controls. Scale bars (F) represents 40 μm.

FIG. 8: Relative signal decrease in EAE animals over the duration of disease progression as detected by platelet targeted SPIOs. (n=3 for days 7, 10 and 14; n=2 for day 17; * p<0.05, ** p<0.002).

FIGS. 9A-9B: Staining of the vasculature in the cerebellum of mice with EAE on day 7 after injection of targeted SPIOs (B) compared to the infusion of non-targeted SPIOs (A) resulting in an image in (B) that is granier (similar to pixilation or small diffuse and scattered dark spots) compared with (A) which is smoother and more contiguous in comparison.

### DETAILED DESCRIPTION OF THE INVENTION

The inventors have selectively targeted activated platelets using a single-chain antibody that recognizes ligand-induced binding sites (LIBS) of GPIIb/IIIa, as disclosed in international patent application no. PCT/AU2006/000943, published as WO 2007/003010, the entire contents of which are incorporated herein by reference. The GPIIb/IIIa epitope becomes exposed only upon activation through receptor-ligand binding, and therefore offers the opportunity to target activated platelets, such as are found on damaged endothelium caused by inflammation or atherosclerotic plaque rupture.

This single chain Fv antibody (scFv) is conjugated to microparticles of iron oxide (MPIO) to create an activation-specific platelet compound (LIBS-MPIO) that may be used with known imaging technology, for example, MRI. Alternatively, the scFv is conjugated to super paramagnetic iron oxide (SPIO) particles to create an activation-specific platelet compound (LIBS-SPIO) that may equally be used with known imaging technology, for example, MRI. The LIBS-MPIO has been used in an animal model to detect vascular platelet aggregation associated with cerebral malaria before pathology is visible by conventional in vivo MRI. The inventors further demonstrate that platelet accumulation is induced in the brain microvasculature by the proinflammatory cytokine TNF, but not by either IL-1β or LT-α. Following platelet accumulation, TNF, but not IL-1β, also induced the adherence of mononuclear cells to the cerebral vasculature in a manner that is indicative of CM pathology. In addition, the inventors have used the LIBS-SPIO in an animal model of multiple sclerosis to demonstrate early detection of experimental autoimmune encephalomyelitis (EAE) in mice using magnetic resonance imaging (MRI).

The inventors have therefore used multiple animal models closely resembling human pathologies to demonstrate that accumulation of platelets in the brain microvasculature can be detected with magnetic resonance imaging (MRI) using a compound at a time when the pathology of the disease is undetectable by conventional MRI. Moreover, the inventors have surprisingly found that such imaging of activated platelets provides a pre-clinical indication of pathology, not only at the very early stages of inflammation, but also at the pre-inflammation stage. This ability to accurately image activated platelets per se, as opposed to imaging downstream consequences of such activation, for example, imaging thrombi, provides an extremely valuable advantage because it facilitates diagnosis and treatment for pathologies before the consequences of inflammation are manifested.

Ligand-induced binding sites (LIBS) on activated platelet GPIIb/IIIa receptors were detected in malaria-infected mice, six days after inoculation with Plasmodium berghei ANKA-pRBC, using a single-chain antibody conjugated to microparticles of iron oxide (LIBS-MPIO). Relatively little or substantially no binding of the LIBS-MPIO compound was detected in control, uninfected animals. A combination of MRI techniques using this compound, confocal microscopy, and transmission electron microscopy revealed that the proinflammatory cytokine TNF, but not IL-1β or LT-α, induces adherence of platelets to cerebrovascular endothelium. Peak platelet adhesion was found 12 h after TNF injection and was readily detected with LIBS-MPIO contrast-enhanced MRI. Temporal studies revealed that the level of MPIO-induced contrast was proportional to the number of platelets bound. Thus, the LIBS-MPIO agent enabled non-invasive detection of otherwise undetectable disease pathology by in vivo MRI before the appearance of overt clinical signs. These results highlight the potential of specific targeted contrast agents for diagnostic, mechanistic and therapeutic applications.

Disclosed herein is therefore the application of functional activation-specific platelet compounds (LIBS-MPIO and LIBS-SPIO) for the detection of vascular platelet adhesion in vivo. The use of these compounds has contributed significantly to understanding of the mechanism of platelet aggregation in the pathology of cerebral malaria and EAE/multiple sclerosis, which the person skilled in the art will appreciate and understand are to be viewed as model diseases. Accordingly, the present disclosure may be applied to a broad range of diseases, including but not limited to stroke, thrombosis, cardiovascular disease, inflammatory diseases, autoimmune diseases, immunoinflammatory diseases, allergic diseases, predispositions thereto, infectious diseases and/or cancer.

The teachings in this disclosure therefore include:

(1) Using the activation-specific LIBS-MPIO and LIBS-SPIO compounds, diagnosis of the pathology of diseases such as cerebral malaria and EAE/multiple sclerosis involving platelet aggregation is possible at an earlier stage than either clinical findings or conventional MRI allow. The functional approach used here enables imaging of activated platelets and platelet thrombi that cannot be detected by routine MRI sequences.

(2) Platelet binding to the brain endothelium is cytokine-specific and TNF is the principal mediator in CM.

(3) After the appearance of platelets in the microvasculature TNF, but not IL-1β, induces the adherence of mononuclear cells to the cerebral vasculature in a manner indicative of CM pathology.

(4) The molecular imaging strategy applying LIBS-MPIO and LIBS-SPIO may be used for the detection of pathologies involving platelets, constituting a non-invasive in vivo examination with high sensitivity and specificity.

Furthermore, the use of micrometer-sized particles for the detection of sparse and difficult-to-access functional epitopes is advantageous, and the excellent contrast properties of the imaging agents disclosed herein have greatly improved the ability to diagnose and predict diseases involving aggregated platelets.

Accordingly, another important advantage of the present invention is the use of a single-chain antibody in the LIBS-MPIO and LIBS-SPIO contrast agents, thus enabling binding to inaccessible targets such as ligand-induced binding sites on activated GPIIb/IIIa. Single-chain antibodies are also less immunogenic than IgG-sized proteins due to the lack of the Fc-regions, and in spite of a small protein size allow the attachment of micrometer-sized iron oxide particles to target epitopes as shown in this study.

As disclosed herein, differential binding patterns of the LIBS-MPIO compound were evident in each CM experimental model. In TNF-injected animals, compound binding was observed in both hemispheres encompassing both cortical and striatal regions, although more contrast was evident in the injected (left) hemisphere. Immunohistochemically, adhesion molecule expression and platelet adhesion was found to be bilateral at the time point used in the current study, although in accord with the MRI findings more staining was present in the left hemisphere. Conversely, in CM animals cortical binding was more pronounced than subcortical contrast changes. Maximum binding of LIBS-MPIO to platelets in the microvasculature is highest at 12 h as detected by MRI, correlating to the number of platelets present. This suggests that increased platelet load over time is directly reflected by increased LIBS-MPIO induced signal void. The correlation is approximate, and the person skilled in the art would not expect perfect stoichiometry between the number of platelets and bound particles. The local environment—size of vessel, presence of other leukocytes, etc—are all likely to affect both binding and changes in signal intensity. For example, once a number of LIBS-MPIO have bound, and generated a signal void, any subsequent binding cannot reduce the signal any further at that location. In the clinical arena, where detecting the spatial distribution of the platelets is of primary interest, this issue is unlikely to be of significance

The concept of functional glycoprotein IIb/IIIa targeting opens up the possibility of detecting and imaging in vivo platelet aggregation such as is found in post-mortem tissue from the brains of patients with CM, multiple sclerosis, HIV-dementia, and bacterial meningitis. The potential applications of this or similar compounds are therefore far reaching. In particular, the use of such an agent for monitoring antiplatelet therapy may have considerable utility across a number of diseases, including rheumatoid arthritis, stroke, thrombosis and cardiovascular disease, as well as in CM as described here.

Another important property for the purposes of therapy monitoring is the biodistribution of the particles as well as the duration of compound binding to the targeted receptors. In animals imaged for the first time 6 h after intracerebral TNF injection, the inventors have discovered that the signal from the LIBS-MPIO in the brains of these mice is lost completely after 10 hours as detected by a second scan. This suggests degradation of the contrast agent independent upon presence of platelets, which is an important prerequisite for serial imaging.

In vivo treatment with a mAb-to-lymphocyte function-associated antigen-1 (LFA-1), which is expressed on platelets and binds ICAM-1, selectively abrogates the cerebral sequestration of platelets, and prevents the development of CM. However, a broad spectrum of proinflammatory agents rapidly upregulates the adhesion molecule ICAM-1 on the cerebral endothelium in a time course that is comparable with that demonstrated on non-CNS endothelium. Leukocyte recruitment is often negligible, and little is known about the adhesion of platelets in these models. IL-1β and TNF both upregulate ICAM-1 on the brain endothelium, and, therefore, the differential binding of platelets to the brain endothelium following the microinjection of these cytokines into the brain was surprising and unexpected. This suggests that while LFA-1/ICAM-1 interactions are undoubtedly important in the adhesion of platelets, they are unlikely to be sufficient, and other factors must be contributing. Non-adhesion molecule-related vascular events, such as cytokine-induced volume changes, may also play a role. It is of interest to note that monocyte recruitment follows platelet adhesion and suggests that the adhesion of platelets provides a scaffold for the subsequent recruitment of monocytes, which happens at a time when regional blood flow has returned to normal.

The present invention therefore teaches that in experimental models of human neurological diseases, targeted contrast agents can be used to detect pathology earlier than conventional, clinically used, MRI approaches or clinical assessment. Wall-adherent platelets were detected non-invasively in vivo in a model of cerebral malaria, prior to the onset of clinical symptoms, using a functional MRI contrast agent, which targets ligand-induced binding sites on activated glycoprotein IIb/IIIa-receptors. In contrast, conventional MRI techniques failed to reveal the presence of CNS pathology before the appearance of overt clinical signs. Owing to the high specificity and sensitivity of the LIBS-MPIO compound, the inventors have for the first time identified TNF as the mediator responsible for platelet aggregation. Furthermore, the inventors were able to demonstrate that due to disappearance of the contrast agent signal over time, serial imaging is possible. This disclosure demonstrates the potential of such agents in enabling the non-invasive assessment of pathological mechanisms in disease models for diagnostic purposes, mechanistic studies and monitoring of therapy.

Methods for Diagnosis, Monitoring and Predicting Disease and Responsiveness to Therapy

The present invention provides methods for diagnosing or predicting a disease in a subject, wherein the method comprises administering to the subject a compound comprising a binding element capable of specifically binding to an activated platelet and an imaging agent, wherein binding of the compound to an activated platelet is indicative of the disease.

The present invention also provides methods for detecting aggregation of platelets in a subject, wherein the method comprises administering to the subject a compound comprising a binding element capable of specifically binding to an activated platelet and an imaging agent, wherein binding of the compound to an activated platelet is indicative of the disease.

The methods may comprise an immunoassay. The immunoassay may comprise magnetic resonance imaging.

The methods may further comprise determining a level of cellular expression of at least one additional molecule.

The at least one additional molecule may be a polynucleotide or a polypeptide encoded thereby.

The polynucleotide may encode tumour necrosis factor (TNF).

The polypeptide may comprise tumour necrosis factor (TNF).

The polypeptide may be intracellular, cell surface-associated or secreted.

The methods may be used for diagnosing, predicting or monitoring stroke, thrombosis, cardiovascular disease, inflammatory diseases, autoimmune diseases, immunoinflammatory diseases, allergic diseases, predispositions thereto, infectious diseases, cancer and/or cancer treatment.

The methods may be used for predicting or monitoring responses to therapy for stroke, thrombosis, cardiovascular disease, inflammatory diseases, autoimmune diseases, immunoinflammatory diseases, allergic diseases, predispositions thereto, infectious diseases and/or cancer.

The inflammatory diseases may include cerebral malaria, multiple sclerosis, Alzheimer's disease, Parkinson's disease, dementia, rheumatoid arthritis, atherosclerosis, unstable plaques and sepsis.

Methods for Treating Disease and TNF Inhibitors

The present invention provides methods for treating diseases in subjects, wherein the method comprises administering to the subject a compound comprising a binding element capable of specifically binding to an activated platelet and an agent that inhibits TNF, wherein binding of the compound to an activated platelet facilitates inhibition of TNF signaling by the agent.

Two strategies for directly inhibiting TNF that have been extensively studied consist of monoclonal anti-TNF antibodies and soluble TNF receptors (sTNF-R). Inhibitors of TNF are therefore known to those of skill in the art and may include Infliximab Remicade® (Mouse-human chimeric anti-huTNF mAb), D2E7 (Humira™) (Fully human anti-huTNF mAb), Etanercept (Enbrel®) (p75sTNF-RII-Fc (dimeric)), PEG-p55sTNF-RI (monomeric) and Lenercept (p55sTNF-RI-IgG1 (dimeric)).

Alternatively, TNF receptors or their ligands may be targeted by the agents herein so as to effect inhibition of TNF signaling. TNF receptors are well known to those of skill in the art and include ligand Lymphotoxin-a and TNF receptor TNF-R1 and -RII, ligand TNF-a and TNF receptor TNF-RI and -RII, ligand Lymphotoxin-b and TNF receptor LT-bR, ligand OX40L and TNF receptor OX40, ligand CD40L and TNF receptor CD40, ligand FasL and TNF receptor Fas, ligand CD27L and TNF receptor CD27, ligand CD30L and TNF receptor CD30, and ligand 4-1BB and TNF receptor 4-1BB. Such targeting may involve the use of antibodies specific for these ligands or receptors, with methods for the production of such antibodies being known to those of skill in the art, as herein described.

Kits

The kits of the present invention facilitate the employment of methods of the invention. Typically, kits for carrying out a method of the invention contain all the necessary reagents to carry out the method. For example, in one embodiment the kits may comprise a first container containing binding agent such as an antibody or a variant, fragment, analogue or derivative thereof, and a second container containing an imaging agent such as a conjugate comprising a binding partner of the antibody, together with a detectable label.

Typically, the kits described above will also comprise one or more other containers, containing for example, wash reagents, and/or other reagents capable of quantitatively detecting the presence of bound antibodies. The detection reagents may include labelled (secondary) antibodies or, where the antibody raised against an antigen is itself labelled, the compartments may comprise antibody binding reagents capable of reacting with the labelled antibody.

In the context of the present invention, a compartmentalised kit includes any kit in which reagents are contained in separate containers, and may include small glass containers, plastic containers or strips of plastic or paper. Such containers may allow the efficient transfer of reagents from one compartment to another compartment whilst avoiding cross-contamination of the samples and reagents, and the addition of agents or solutions of each container from one compartment to another in a quantitative fashion. Such kits may also include a container which will accept the test sample, a container which contains the antibody(s) used in the assay, containers which contain wash reagents (such as phosphate buffered saline, Tris-buffers, and like), and containers which contain the detection reagent.

Typically, a kit of the present invention will also include instructions for using the kit components to conduct the appropriate methods.

Antibodies

Particular embodiments of the invention provide for the use of one or more antibodies or variants, fragments, analogues or derivatives thereof, for the detection of activated platelets. Antibodies suitable for use in the methods of the present invention can be raised against target antigens using techniques known to those in the art. Suitable antibodies include, but are not limited to polyclonal, monoclonal, chimeric, humanised, single chain (sc), scFv, Fab fragments, and a Fab expression library. Suitable antibodies may be prepared from discrete regions or fragments of target antigens. An antigenic polypeptide contains at least about 5, and typically at least about 10, amino acids. Methods for the generation of suitable antibodies will be readily appreciated by those skilled in the art. For example, a monoclonal antibody, typically containing Fab portions, may be prepared using the hybridoma technology described in Antibodies—A Laboratory Manual, Harlow and Lane, eds., Cold Spring Harbor Laboratory, N.Y. (1988). In the preparation of monoclonal antibodies directed toward a polypeptide, fragment or analogue thereof, any technique that provides for the production of antibody molecules by continuous cell lines in culture may be used. These include the hybridoma techniques originally developed by Kohler et al., Nature, 256:495-497 (1975), as well as the trioma technique, the human B-cell hybridoma technique [Kozbor et al., Immunology Today, 4:72 (1983)], and the EBV-hybridoma technique to produce human monoclonal antibodies [Cole et al., in Monoclonal Antibodies and Cancer Therapy, pp. 77-96, Alan R. Liss, Inc., (1985)]. Immortal, antibody-producing cell lines can be created by techniques other than fusion, such as direct transformation of B lymphocytes with oncogenic DNA, or transfection with Epstein-Barr virus. See, e.g., M. Schreier et al., “Hybridoma Techniques” (1980); Hammerling et al., “Monoclonal Antibodies and T-cell Hybridomas” (1981); Kennett et al., “Monoclonal Antibodies” (1980).

A monoclonal antibody useful in practicing the present invention can be produced by initiating a monoclonal hybridoma culture comprising a nutrient medium containing a hybridoma that secretes antibody molecules of the appropriate antigen specificity. The culture is maintained under conditions and for a time period sufficient for the hybridoma to secrete the antibody molecules into the medium. The antibody-containing medium is then collected. The antibody molecules can then be further isolated by well-known techniques.

Similarly, there are various procedures known in the art which may be used for the production of polyclonal antibodies, or variants, fragments or analogues thereof. For the production of polyclonal antibodies, various host animals can be immunized by injection with a polypeptide, or a variant, fragment or analogue thereof, including but not limited to rabbits, mice, rats, sheep, goats, etc. Further, a polypeptide or variant, fragment or analogue thereof can be conjugated to an immunogenic carrier, e.g., bovine serum albumin (BSA) or keyhole limpet hemocyanin (KLH).

Screening for an antibody can also be accomplished by a variety of techniques known in the art. Assays for immunospecific binding of antibodies may include, but are not limited to, radioimmunoassays, ELISAs (enzyme-linked immunosorbent assay), sandwich immunoassays, immunoradiometric assays, gel diffusion precipitation reactions, immunodiffusion assays, in situ immunoassays, Western blots, precipitation reactions, agglutination assays, complement fixation assays, immunofluorescence assays, protein A assays, and immunoelectrophoresis assays (see, for example, Ausubel et al., eds, 1994, Current Protocols in Molecular Biology, Vol. 1, John Wiley & Sons, Inc., New York). Antibody binding may be detected by virtue of a detectable label on a primary antibody. Alternatively, an antibody may be detected by virtue of its binding with a secondary antibody or reagent which is appropriately labeled. A variety of methods are known in the art for detecting antibody binding in an immunoassay and these methods are within the scope of the present invention.

In terms of obtaining a suitable amount of an antibody according to the present invention, one may manufacture the antibody(ies) using batch cell culture with serum free medium. After cell culture, the antibody may be purified via a multistep procedure incorporating chromatography and viral inactivation/removal steps. For instance, the antibody may be first separated by Protein A affinity chromatography and then treated with solvent/detergent to inactivate any lipid-enveloped viruses. Further purification, typically by anion and cation exchange chromatography may be used to remove residual proteins, solvents/detergents and nucleic acids. The crudely purified antibody may be further purified and formulated into 0.9% saline using gel filtration columns. The formulated bulk preparation may then be sterilised and viral filtered and dispensed.

In another embodiment of the present invention, the cell may be an insect cell infected with a recombinant baculovirus, wherein the baculovirus contains an expression cassette into which has been cloned a polynucleotide encoding a desired polypeptide. Such baculovirus expression systems are well known to those of skill in the art. Kits and services facilitating the construction of such baculovirus expression systems are commercially available and include, for example, the BD BaculoGold™ Baculovirus Expression Vector System (BD Biosciences, United States of America) and Invitrogen's Baculovirus Expression Services (Invitrogen, United States of America).

In preferred embodiments of the present invention, the antibody may be of a size in the range of 2 kDa to 100 kDa, 4 kDa to 90 kDa, 6 kDa to 80 kDa, 8 kDa to 70 kDa, 10 kDa to 60 kDa, 12 kDa to 58 kDa, 14 kDa to 56 kDa, 16 kDa to 54 kDa, 18 kDa to 52 kDa, 20 kDa to 50 kDa, 21 kDa to 48 kDa, 22 kDa to 46 kDa, 23 kDa to 44 kDa, 24 kDa to 42 kDa, 25 kDa to 40 kDa, 26 kDa to 38 kDa, 27 kDa to 36 kDa, 29 kDa to 34 kDa, 30 kDa to 33 kDa or 32 kDa. As a relatively small sized antibody has an advantage of being able to circulate to microvasculature in a subject, the antibody may be a single chain Fv antibody.

Imaging Agents and Imaging Techniques

Imaging agents suitable for use in the present invention include those agents useful in imaging molecular complexes or labelled tissues in vitro, ex vivo, or in vivo. By way of non-limiting example, agents include those disclosed in WO 2007/099289; U.S. Pat. No. 7,029,655; U.S. Pat. Nos. 6,627,176; and 6,911,457, examples of which would be suitable and may be adapted to being conjugated to the binding elements of the invention. In vivo labels or indicating means are those useful within the body of a vertebrate.

The linking of labels, i.e., labelling of, polypeptides and proteins is well known in the art. For instance, antibody molecules produced by a hybridoma can be labeled by metabolic incorporation of radioisotope-containing amino acids provided as a component in the culture medium. See, for example, Galfre et al., Meth. Enzymol., 73:3-46 (1981). The techniques of protein conjugation or coupling through activated functional groups are particularly applicable. See, for example, Aurameas, et al., Scand. J. Immunol., Vol. 8, Suppl. 7:7-23 (1978), Rodwell et al., Biotech., 3:889-894 (1984), and U.S. Pat. No. 4,493,795. Methods of conjugating appropriate imaging agents to the binding elements of the invention are well known in the art. See, for example, WO 2007/099289 and the references cited therein. Again, choice of conjugation method will depend upon the imaging agent chosen and the binding element chosen.

The techniques of imaging useful in the invention include any technique capable of detecting and/or generating a processable signal that identifies the presence or location of the imaging agents incorporated in the invention. Exemplary imaging techniques include in vivo NMR Imaging and X-ray imaging, though any technique capable of detecting and thereby imaging the imaging agents used in the invention are contemplated. Of course, the choice of imaging agent will be influenced by or influence the choice of imaging technique.

By way of example, nuclear magnetic resonance (NMR) is now widely used for obtaining spatial images of human subjects for clinical diagnosis. Clinical usage of NMR imaging, also called magnetic resonance imaging or, simply, MRI, for diagnostic purposes has been reviewed [see e.g., Pykett, et al., Nuclear Magnetic Resonance, pgs. 157-167 (April, 1982) and T. F. Budinger, et al., Science, pgs. 288-298, (October, 1984)]. Several distinctive characteristics of using such a procedure over other useful diagnostic methods, e.g., x-ray computer-aided tomography (CT), are generally recognized. For instance, the magnetic fields utilized in a clinical NMR scan are not considered to possess any deleterious effects to human health. Additionally, while x-ray CT images are formed from the observation of a single parameter, x-ray attenuation, MR images are a composite of the effects of a number of parameters which are analyzed and combined by computer. Choice of the appropriate instrument parameters such as radio frequency (Rf), pulsing and timing can be utilized to enhance (or, conversely, attenuate) the signals of any of the image-producing parameters thereby improving the image quality and providing better anatomical and functional information. Finally, the use of such imaging has, in some cases, proven to be a valuable diagnostic tool as normal and diseased tissue, by virtue of their possessing different parameter values, can be differentiated in the image.

In MRI, the image of an organ or tissue is obtained by placing a subject in a strong external magnetic field and observing the effect of this field on the magnetic properties of the protons (hydrogen nuclei) contained in and surrounding the organ or tissue. The proton relaxation times, termed T₁ and T₂, are of primary importance. T₁ (also called the spin-lattice or longitudinal relaxation time) and T₂ (also called the spin-spin or transverse relaxation time) depend on the chemical and physical environment of organ or tissue protons and are measured using the Rf pulsing technique; this information is analyzed as a function of distance by computer which then uses it to generate an image.

The image produced, however, often lacks definition and clarity due to the similarity of the signal from other tissues. To generate an image with good definition, T₁ and/or T₂ of the tissue to be imaged must be distinct from that of the background tissue. In some cases, the magnitude of these differences is small, limiting diagnostic effectiveness. Thus, there exists a real need for methods which increase or magnify these differences. One approach is the use of contrast agents.

Since any material suitable for use as a contrast agent must affect the magnetic properties of the surrounding tissue, MRI contrast agents can be categorized by their magnetic properties.

Paramagnetic materials have been used as MRI contrast agents because of their long recognized ability to decrease T₁ [Weinmann et al., Am. J. Rad. 142, 619 (1984), Greif et al. Radiology 157, 461 (1985), Runge, et al. Radiology 147, 789 (1983), Brasch, Radiology 147, 781 (1983)]. Paramagnetic materials are characterized by a weak, positive magnetic susceptibility and by their inability to remain magnetic in the absence of an applied magnetic field.

Paramagnetic MRI contrast agents are usually transition metal ions of iron, manganese or gadolinium. They may be bound with chelators to reduce the toxicity of the metal ion. Paramagnetic materials for use as MRI contrast agents are the subject of a number of patents and patent applications. (See EPA 0 160 552; UK Application 2 137 612A; EPA 0 184 899; EPA 0 186 947; U.S. Pat. No. 4,615,879; PCT WO 85/05554; and EPA 0 210 043).

Ferromagnetic materials have also been used as contrast agents because of their ability to decrease T₂ [Medonca-Dias and Lauterbur, Magn. Res. Med. 3, 328, (1986); Olsson et al., Mag Res. Imaging 4, 437 (1986); Renshaw et al. Mag Res. Imaging 4, 351 (1986) and 3, 217 (1986)]. Ferromagnetic materials have high, positive magnetic susceptibilities and maintain their magnetism in the absence of an applied field. Ferromagnetic materials for use as MRI contrast agents are the subject of patent applications [PCT WO No. 86/01112; PCT WO No. 85/043301].

A third class of magnetic materials termed superparamagnetic materials have been used as contrast agents [Saini et al., Radiology, 167, 211 (1987); Hahn et al., Soc. Mag Res. Med. 4(22) 1537 (1986)]. Like paramagnetic materials, superparamagnetic materials are characterized by an inability to remain magnetic in the absence of an applied magnetic field. Superparamagnetic materials can have magnetic susceptibilities nearly as high as ferromagnetic materials and far higher than paramagnetic materials [Bean and Livingston J. Appl. Phys. suppl to vol. 30, 1205, (1959)].

Ferromagnetism and superparamagnetism are properties of lattices rather than ions or gases. Iron oxides such as magnetite and gamma ferric oxide exhibit ferromagnetism or superparamagnetism depending on the size of the crystals comprising the material, with larger crystals being ferromagnetic [G. Bate in Ferromagnetic Materials. vol. 2, Wohlfarth (ed.) p. 439].

As generally used, superparamagnetic and ferromagnetic materials alter the MR image by decreasing T₂, resulting in image darkening. When injected, crystals of these magnetic materials accumulate in the targeted organs or tissues and darken the organs or tissues where they have accumulated. In the context of the invention, the binding element acts to specifically localise, and thereby relatively accumulate the signalling agent such that its presence and/or location is detectable by MRI, for example.

Superparamagnetic materials possess some characteristics of paramagnetic and some characteristics of ferromagnetic materials. Like paramagnetic materials, superparamagnetic materials rapidly lose their magnetic properties in the absence of an applied magnetic field; they also possess the high magnetic susceptibility and crystalline structure found in ferromagnetic materials. Iron oxides such as magnetite or gamma ferric oxide exhibit superparamagnetism when the crystal diameter falls significantly below that of purely ferromagnetic materials.

For cubic magnetite (Fe₃O₄) this cut-off is a crystal diameter of about 300 angstroms [Dunlop, J. Geophys. Rev. 78 1780 (1972)]. A similar cut-off applies for gamma ferric oxide [Bare in Ferromagnetic Materials, vol. 2, Wohfarth (ed.) (1980) p. 439]. Since iron oxide crystals are generally not of a single uniform size, the average size of purely ferromagnetic iron oxides is substantially larger than the cut-off of 300 angstroms (0.03 microns). For example, when gamma ferric oxide is used as a ferromagnetic material in magnetic recording, (e.g., Pfizer Corp. Pf 2228), particles are needle-like and about 0.35 microns long and 0.06 microns thick. Other ferromagnetic particles for data recording are between 0.1 and 10 microns in length [Jorgensen, The Complete Handbook of Magnetic Recording, p. 35 (1980)]. For a given type of crystal, preparations of purely ferromagnetic particles have average dimensions many times larger than preparations of superparamagnetic particles.

The theoretical basis of superparamagnetism has been described in detail by Bean and Livington [J. Applied Physics, Supplement to volume 30, 1205 (1959)]. Fundamental to the theory of superparamagnetic materials is the destabilizing effect of temperature on their magnetism. Thermal energy prevents the alignment of the magnetic moments present in superparamagnetic materials. After the removal of an applied magnetic field, the magnetic moments of superparamagnetic materials still exist, but are in rapid motion, causing a randomly oriented or disordered magnetic moment and, thus, no net magnetic field. At the temperatures of biological systems and in the applied magnetic fields of MR imagers, superparamagnetic materials are less magnetic than their ferromagnetic counterparts. For example, Berkowitz et al. [J. App. Phys. 39, 1261 (1968)] have noted decreased magnetism of small superparamagnetic iron oxides at elevated temperatures. This may in part explain why workers in the field of MR imaging have looked to ferromagnetic materials as contrast agents on the theory that the more magnetic a material is per gram, the more effective that material should be in depressing T₂ [Drain, Proc. Phys. Soc. 80, 1380 (1962); Medonca-Dias and Lauterur, Mag. Res. Med. 3, 328 (1986)].

It has been recognized for some time that superparamagnetic particles can be fashioned into magnetic fluids termed ferrofluids [see Kaiser and Miskolczy, J. Appl. Phys. 41 3 1064 (1970)]. A ferrofluid is a solution of very fine magnetic particles kept from settling by Brownian motion. To prevent particle agglomeration through Van der Waals attractive forces, the particles are coated in some fashion. When a magnetic field is applied, the magnetic force is transmitted to the entire volume of liquid and the ferrofluid responds as a fluid, i.e. the magnetic particles do not separate from solvent.

Another approach to synthesizing water-based magnetic compounds is disclosed by Gable et al (U.S. Pat. No. 4,001,288). Here, the patent discloses that magnetite can be reacted with a hydroxycarboxylic acid to form a water soluble complex that exhibits ferromagnetic behavior both in the solid form and in solution.

The manufacture of a magnetic pharmaceutical solution such as an MRI contrast agent requires an extremely stable solution so certain manipulations, common in pharmaceutical manufacture, can be carried out. Solution stability is defined as the retention of the size of the magnetic material in solution; in an unstable solution the material will clump or aggregate. Such changes in the size of magnetic material alter its biodistribution after injection, an intolerable situation for an MRI contrast agent. A high degree of stability is required to perform common operations associated with pharmaceutical manufacture such as dialysis, concentration, filtration, centrifugation, storage of concentrates prior to bottling, and long term storage after bottling. Particular problems are posed by the need to sterilize aqueous solutions of metal oxide, e.g. iron oxide, for pharmaceutical use.

In particular aspects, this invention provides an in vivo MR imaging technique for diagnostic purposes which will produce a clear, well-defined image of the targeted platelets, plaques, lesions, tissues, etc. The agents are easily administered, exert a significant effect on the image produced and localize in vivo to the specific targets. These agents can be easily processed for in vivo use, and overcome problems of toxicity and excessively long retention in the subject (i.e. are biodegradable). In particular embodiments, the imaging agents are biodegradable superparamagnetic metal oxides. Such materials combine an optimal balance of features and are particularly well-suited for use in the invention. Remarkably, it has been found that these agents produce a well-resolved, negative contrast image of the in vivo target. It has also been surprisingly found that the materials used in the methods of this invention exhibit highly desirable in vivo retention times, i.e., they remain intact for a sufficient time to permit the image to be taken, yet are ultimately biodegradable. Remarkably, once degraded, iron-based materials serve as a source of nutritional iron. Additionally, they are sufficiently small to permit free circulation through the subject's vascular system and rapid absorption by the organ/tissue being imaged, allowing for maximum latitude in the choice of administration routes and ultimate targets. In particular, the agents of the invention are sufficiently small (as described herein) to provide surprisingly effective penetration into plaques and early stage platelet aggregations in fine vascular tissues, particularly of the vertebrate brain.

There is a rapidly growing body of literature demonstrating the clinical effectiveness of paramagnetic contrast agents (currently 8 are in clinical trials or in use). The capacity to differentiate regions/tissues that may be magnetically similar but histologically distinct is a major impetus for the preparation of these agents [1,2]. In the design of MRI agents, strict attention must be given to a variety of properties that will ultimately effect the physiological outcome apart from the ability to provide contrast enhancement [3]. Two fundamental properties that must be considered are biocompatability and proton relaxation enhancement. Biocompatability is influenced by several factors including toxicity, stability (thermodynamic and kinetic), pharmacokinetics and biodistribution. Proton relaxation enhancement (or relaxivity) is chiefly governed by the choice of metal and rotational correlation times.

For example, regions associated with a Gd³⁺ ion (near-by water molecules) appear bright in an MR image where the normal aqueous solution appears as dark background if the time between successive scans in the experiment is short (i.e. T₁ weighted image). Localized T₂ shortening caused by superparamagnetic particles is believed to be due to the local magnetic field inhomogeneities associated with the large magnetic moments of these particles. Regions associated with a superparamagnetic iron oxide particle appear dark in an MR image where the normal aqueous solution appears as high intensity background if the echo time (TE) in the spin-echo pulse sequence experiment is long (i.e. T₂-weighted image). The lanthanide atom Gd³⁺ is by the far the most frequently chosen metal atom for MRI contrast agents because it has a very high magnetic moment (u²=63BM²), and a symmetric electronic ground state, (S⁸). Transition metals such as high spin Mn(II) and Fe(III) are also candidates due to their high magnetic moments.

Once the appropriate metal has been selected, a suitable ligand or chelate must be found to render the complex nontoxic. The term chelator is derived from the Greek word chele which means a “crabs claw”, an appropriate description for a material that uses its many “arms” to grab and hold on to a metal atom (see DTPA below). Several factors influence the stability of chelate complexes include enthalpy and entropy effects (e.g. number, charge and basicity of coordinating groups, ligand field and conformational effects). Various molecular design features of the ligand can be directly correlated with physiological results. For example, the presence of a single methyl group on a given ligand structure can have a pronounced effect on clearance rate.

Diethylenetriaminepentaacetic (DTPA) chelates and thus acts to detoxify lanthanide ions. The stability constant (K) for Gd(DTPA)²- is very high (log K=22.4) and is more commonly known as the formation constant (the higher the log K, the more stable the complex). This thermodynamic parameter indicates the fraction of Gd³⁺ ions that are in the unbound state will be quite small and should not be confused with the rate (kinetic stability) at which the loss of metal occurs. The water soluble Gd(DTPA)²- chelate is stable, nontoxic, and one of the most widely used contrast enhancement agents in experimental and clinical imaging research. It was approved for clinical use in adult patients in June of 1988.

To date, a number of chelators have been used, including diethylenetriaminepentaacetic (DTPA), 1,4,7,10-tetraazacyclododecane3-N,N′N″,N′″-tetracetic acid (DOTA), and derivatives thereof. See U.S. Pat. Nos. 5,155,215, 5,087,440, 5,219,553, 5,188,816, 4,885,363, 5,358,704, 5,262,532, and Meyer et al., Invest. Radiol. 25: S53 (1990).

Image enhancement improvements using Gd(DTPA) are well documented in a number of applications (Runge et al., Magn, Reson. Imag. 3:85 (1991); Russell et al., AJR 152:813 (1989); Meyer et al., Invest. Radiol. 25: S53 (1990)) including visualizing blood-brain barrier disruptions caused by space occupying lesions and detection of abnormal vascularity. It has recently been applied to the functional mapping of the human visual cortex by defining regional cerebral hemodynamics (Belliveau et al., (1991) 254:719). Since uncomplexed gadolinium is very toxic, gadolinium chelate probes, such as gadolinium diethylenetriamine pentaacetic acid (GdDTPA MW 570 Da), albumin-GdDTPA (Gadomer-17, MW 35 or 65 kDa), have been employed extensively in MRI.

Another chelator used in Gd contrast agents is the macrocyclic ligand 1,4,7,10-tetraazacyclododecane-N,N′,N″N′″-tetracetic acid (DOTA). The Gd-DOTA complex has been thoroughly studied in laboratory tests involving animals and humans. The complex is conformationally rigid, has an extremely high formation constant (log K=28.5), and at physiological pH possess very slow dissociation kinetics. Recently, the GdDOTA complex was approved as an MRI contrast agent for use in adults and infants in France and has been administered to over 4500 patients.

Attempts have also been made to overcome the low relaxivities of small Gd-DTPA chelates by preparing polymer conjugates of Gd(DTPA)⁽²⁻⁾ [see e.g., MRA. Duarte M. G.; Gil M. H.; Peters J. A.; Colet J. M.; Elst L. Vander; Muller R. N.; Geraldes C. F. G. C., Bioconjug. Chem., 21, 170-177, 2001.]. Although the relaxivity of these polymer conjugates was only slightly improved, they were also cleared very quickly from the blood of rats, indicating that they are of value as contrast agents for MRI where monitoring of therapy is contemplated. The clinical use of polymer-coated paramagnetic iron oxide particles as a tissue-specific MRI contrast agent is well established (R. Weissleder, et al., Radiology, 175, 494-498, 1990.). MRI with iron-oxide particles has been successfully used to image apoptic cells (M. Zhao et al., Nature Medicine, 7, 1241-1244, 2001.) and rat T-cells at the cellular level (S. J. Dodd et al., Biophysical J., 76, 103-109, 1999.)

Methods of Making Binding Elements

An additional path to binding elements of the present invention may be adapted from the techniques described in US Patent Publication No. 20070218067, especially at paragraphs 44 through 130, which are specifically incorporated herein by reference. If the binding element of the invention is made through these referenced techniques, however, there is the additional aspect that whatever binding element is made must not substantially contribute or interfere with fibrinogen binding.

Pharmaceutical Compositions

The compounds, or variants, fragments or analogues thereof as described above, or a combination thereof, may be used with pharmaceutically acceptable diluents, carriers, excipients and/or adjuvants in compositions for diagnosis and therapies as disclosed herein.

Antibodies and other compounds of the present invention may be administered as compositions diagnostically, therapeutically or preventively. In a therapeutic application, compositions are administered to a patient already suffering from a disease, in an amount sufficient to cure or at least partially arrest the disease and its complications. The composition should provide a quantity of the compound or agent sufficient to effectively treat the patient.

In general, suitable compositions may be prepared according to methods which are known to those of ordinary skill in the art and accordingly may include a pharmaceutically acceptable carrier, diluent and/or adjuvant.

Methods for preparing administrable compositions are apparent to those skilled in the art, and are described in more detail in, for example, Remington's Pharmaceutical Science, 15th ed., Mack Publishing Company, Easton, Pa., hereby incorporated by reference herein.

Compositions of the present invention may include topical formulations and comprise an active ingredient together with one or more acceptable carriers, diluents, excipients and/or adjuvants, and optionally any other therapeutic ingredients. Formulations suitable for topical administration include liquid or semi-liquid preparations suitable for penetration through the skin to the site of where treatment is required, such as liniments, lotions, creams, ointments or pastes, and drops suitable for administration to the eye, ear or nose.

Drops according to the present invention may comprise sterile aqueous or oily solutions or suspensions. These may be prepared by dissolving the active ingredient in an aqueous solution of a bactericidal and/or fungicidal agent and/or any other suitable preservative, and optionally including a surface active agent. The resulting solution may then be clarified by filtration, transferred to a suitable container and sterilised. Sterilisation may be achieved by: autoclaving or maintaining at 90° C.-100° C. for half an hour, or by filtration, followed by transfer to a container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions according to the present invention include those suitable for application to the skin or eye. An eye lotion may comprise a sterile aqueous solution optionally containing a bactericide and may be prepared by methods similar to those described above in relation to the preparation of drops. Lotions or liniments for application to the skin may also include an agent to hasten drying and to cool the skin, such as an alcohol or acetone, and/or a moisturiser such as glycerol, or oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogels.

The composition may incorporate any suitable surfactant such as an anionic, cationic or non-ionic surfactant such as sorbitan esters or polyoxyethylene derivatives thereof, Suspending agents such as natural gums, cellulose derivatives or inorganic materials such as silicaceous silicas, and other ingredients such as lanolin, may also be included.

The compositions may also be administered in the form of liposomes. Liposomes are generally derived from phospholipids or other lipid substances, and are formed by mono- or multi-lamellar hydrated liquid crystals that are dispersed in an aqueous medium. Any non-toxic, physiologically acceptable and metabolisable lipid capable of forming liposomes can be used. The compositions in liposome form may contain stabilisers, preservatives, excipients and the like. The preferred lipids are the phospholipids and the phosphatidyl cholines (lecithins), both natural and synthetic. Methods to form liposomes are known in the art, and in relation to this specific reference is made to: Prescott, Ed., Methods in Cell Biology, Volume XIV, Academic Press, New York, N.Y. (1976), p. 33 et seq., the contents of which are incorporated herein by reference.

Dosages

The therapeutically effective dose level for any particular patient will depend upon a variety of factors including: the disorder being treated and the severity of the disorder; activity of the compound or agent employed; the composition employed; the age, body weight, general health, sex and diet of the patient; the time of administration; the route of administration; the rate of sequestration of the agent or compound; the duration of the treatment; drugs used in combination or coincidental with the treatment, together with other related factors well known in medicine.

One skilled in the art would be able, by routine experimentation, to determine an effective, non-toxic amount of agent or compound which would be required to treat applicable diseases.

Generally, an effective dosage is expected to be in the range of about 0.0001 mg to about 1000 mg per kg body weight per 24 hours; typically, about 0.001 mg to about 750 mg per kg body weight per 24 hours; about 0.01 mg to about 500 mg per kg body weight per 24 hours; about 0.1 mg to about 500 mg per kg body weight per 24 hours; about 0.1 mg to about 250 mg per kg body weight per 24 hours; about 1.0 mg to about 250 mg per kg body weight per 24 hours. More typically, an effective dose range is expected to be in the range about 1.0 mg to about 200 mg per kg body weight per 24 hours; about 1.0 mg to about 100 mg per kg body weight per 24 hours; about 1.0 mg to about 50 mg per kg body weight per 24 hours; about 1.0 mg to about 25 mg per kg body weight per 24 hours; about 5.0 mg to about 50 mg per kg body weight per 24 hours; about 5.0 mg to about 20 mg per kg body weight per 24 hours; about 5.0 mg to about 15 mg per kg body weight per 24 hours.

Alternatively, an effective dosage may be up to about 500 mg/m². Generally, an effective dosage is expected to be in the range of about 25 to about 500 mg/m², preferably about 25 to about 350 mg/m², more preferably about 25 to about 300 mg/m², still more preferably about 25 to about 250 mg/m², even more preferably about 50 to about 250 mg/m², and still even more preferably about 75 to about 150 mg/m².

Typically, in therapeutic applications, the treatment would be for the duration of the disease state.

Further, it will be apparent to one of ordinary skill in the art that the optimal quantity and spacing of individual dosages will be determined by the nature and extent of the disease state being treated, the form, route and site of administration, and the nature of the particular individual being treated. Also, such optimum conditions can be determined by conventional techniques.

It will also be apparent to one of ordinary skill in the art that the optimal course of treatment, such as the number of doses of the composition given per day for a defined number of days, can be ascertained by those skilled in the art using conventional course of treatment determination tests.

Routes of Administration

The compositions of the present invention can be administered by standard routes. In general, the compositions may be administered by the parenteral (e.g., intravenous, intraspinal, subcutaneous or intramuscular), oral or topical route. Typically, administration is by the intravenous, intramuscular, subcutaneous or intraperitoneal route. The compositions can also be injected directly into the synovial joints or the site of inflammation.

Carriers, Diluents, Excipients and Adjuvants

Carriers, diluents, excipients and adjuvants must be “acceptable” in terms of being compatible with the other ingredients of the composition, and not deleterious to the recipient thereof. Such carriers, diluents, excipient and adjuvants may be used for enhancing the integrity and half-life of the compositions of the present invention. These may also be used to enhance or protect the biological activities of the compositions of the present invention.

Examples of pharmaceutically acceptable carriers or diluents are demineralised or distilled water; saline solution; vegetable based oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrolidone; agar; gum tragacanth or gum acacia, and petroleum jelly. Typically, the carrier or carriers will form from 10% to 99.9% by weight of the compositions.

Other carriers may include viral-vectors in which DNA encoding the compounds of the present invention can be delivered directly into target cells.

The carriers may also include fusion proteins or chemical compounds that are covalently bonded to the compounds of the present invention. Such biological and chemical carriers may be used to enhance the delivery of the compounds to the targets or enhance therapeutic activities of the compounds. Methods for the production of fusion proteins are known in the art and described, for example, in Ausubel et al (In: Current Protocols in Molecular Biology. Wiley Interscience, ISBN 047 150338, 1987) and Sambrook et al (In: Molecular Cloning: A Laboratory Manual, Cold Spring Harbor Laboratories, New York, Third Edition 2001).

The compositions of the invention may be in a form suitable for administration by injection, in the form of a formulation suitable for oral ingestion (such as capsules, tablets, caplets, elixirs, for example), in the form of an ointment, cream or lotion suitable for topical administration, in a form suitable for delivery as an eye drop, in an aerosol form suitable for administration by inhalation, such as by intranasal inhalation or oral inhalation, in a form suitable for parenteral administration, that is, subcutaneous, intramuscular or intravenous injection.

For administration as an injectable solution or suspension, non-toxic parenterally acceptable diluents or carriers can include, Ringer's solution, isotonic saline, phosphate buffered saline, ethanol and 1,2 propylene glycol.

Some examples of suitable carriers, diluents, excipients and/or adjuvants for oral use include peanut oil, liquid paraffin, sodium carboxymethylcellulose, methylcellulose, sodium alginate, gum acacia, gum tragacanth, dextrose, sucrose, sorbitol, mannitol, gelatine and lecithin. In addition these oral formulations may contain suitable flavouring and colourings agents. When used in capsule form the capsules may be coated with compounds such as glyceryl monostearate or glyceryl distearate which delay disintegration.

Adjuvants typically include Freund' adjuvants, emollients, emulsifiers, thickening agents, preservatives, bactericides and buffering agents. Other adjuvants may be used to increase the immunological response, including but not limited to mineral gels such as aluminium hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

Solid forms for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Liquid forms for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Suspensions for oral administration may further comprise dispersing agents and/or suspending agents. Suitable suspending agents include sodium carboxymethylcellulose, methylcellulose, hydroxypropylmethyl-cellulose, poly-vinyl-pyrrolidone, sodium alginate or acetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like.

The emulsions for oral administration may further comprise one or more emulsifying agents. Suitable emulsifying agents include dispersing agents as exemplified above or natural gums such as guar gum, gum acacia or gum tragacanth.

Combinations

Those skilled in the art will appreciate that the compositions may be administered as part of a combination diagnostic or therapy approach, employing one or more of the compositions disclosed herein in conjunction with other therapeutic approaches to such treatment. For such combination therapies, each component of the combination may be administered at the same time, or sequentially in any order, or at different times, so as to provide the desired therapeutic effect. When administered separately, it may be preferred for the components to be administered by the same route of administration, although it is not necessary for this to be so. Alternatively, the components may be formulated together in a single dosage unit as a combination product. Suitable agents which may be used in combination with the compositions of the present invention will be known to those of ordinary skill in the art.

Timing of Therapies

Those skilled in the art will appreciate that the compositions may be administered as a single agent or as part of a combination diagnostic or therapy approach, for example, as a follow-up diagnostic, monitoring, treatment or consolidation therapy as a compliment to currently available therapies for such diseases. The compositions may also be used as preventative therapies for subjects who are genetically or environmentally predisposed to developing such diseases.

The present invention will now be further described in greater detail by reference to the following specific examples, which should not be construed as in any way limiting the scope of the invention.

### EXAMPLES

### Example 1

### General Methods

1.1 Single-Chain Antibody Generation, Conjugation to 1 μm Iron Oxide Microparticles and In Vitro Binding Studies

The monoclonal antibody (mAb) anti-LIBS145 binds to GPIIb/IIIa only in its active conformation, and demonstrates strong binding to ADP-activated platelets in the presence of fibrinogen. Generation of anti-LIBS145 has been described in detail elsewhere (34). In brief, the mAb anti-LIBS-145-expressing hybridoma cell line was used as the basis for the cloning of an anti-LIBS single-chain antibody (scFv). mRNA of this hybridoma cell line was prepared and reverse transcribed using an oligo-dT primer. The variable regions of the antibody's heavy and light chain were amplified by PCR and cloned into the pHOG21 vector, TG1 E. coli. Individual clones were assessed for LIBS-typical binding to GPIIb/IIIa in flow cytometry using activated platelets. Finally, the best binding scFv_{LIBS} was produced in LB media containing ampicillin and glucose. Centrifuged and pelleted bacteria were resuspended in BugBuster® (Novagen) and again centrifuged and the supernatant containing soluble protein was kept on ice after adding a protease inhibitor (Complete® Roche). The supernatant was mixed with Ni²⁺-Agarose (Qiagen) binding His(6)-tagged proteins. Finally, the scFv was eluted at high imidazole concentrations and dialysed to PBS. Functionality of the scFv preparations was evaluated by flow cytometry.

For the irrelevant control antibody, exchange of the arginine in the RXD motif of the heavy chain CDR3 region of a platelet single-chain antibody was performed to achieve a non-functional antibody for control purpose. The generation and purification of this antibody was performed in the same way as described above.

For construction of the contrast agent, autofluorescent cobalt-functionalised MPIOs (1 □m) were conjugated to the histidine-tag of the LIBS/control single-chain antibody referring to the protocol of the manufacturer (Dynal Biotech, Oslo, Norway). In brief, 1 mg of beads was incubated with the LIBS/control antibody for 10 min at room temperature to bind approximately 10 mg of histidine-tagged antibody. The tube containing the suspension was then placed on a magnet until the beads had migrated to the side of the tube and the supernatant was discarded. This washing step was repeated four times using a binding and washing buffer as described by the manufacturer. MPIOs conjugated to the LIBS-antibody will be referred to here as LIBS-MPIO, MPIO conjugated to control antibodies are named control-MPIO.

To examine the binding characteristics of the LIBS-MPIO contrast agent, blood from healthy human volunteers was obtained. After the centrifugation of whole blood (1000 rpm, 10 min), 50 □l of platelet rich plasma was incubated with either 20 □M ADP, a potent platelet activator, or vehicle on a microscope slide. LIBS-MPIO, control-MPIO, or non-functionalized MPIOs were applied and incubated under continuous and careful rotation. After 10 minutes the slides were washed, coverslipped, and MPIO binding was evaluated.

1.2 Murine Malaria Model

Female C57BL6-mice were purchased from Charles River, UK. Infections were initiated by i.p. injection of 10⁶ P. berghei ANKA-pRBC per mouse. Parasitemia and health status were monitored on a daily basis in accordance with our UK Home Office licence. The level of parasitemia was evaluated on blood smears after Giemsa staining.

1.3 Stereotaxic Microinjection of Recombinant Cytokines

The malaria model is mouse specific, but the histological outcomes, following the microinjection of cytokines in the rat or mouse brain is conserved (15, 29, 35, 36). Thus rats or mice were used, depending on the availability of complementary antibodies, to examine the profile of platelet binding and microvessel integrity after the microinjection of cytokine into the brain.

Rat-recombinant TNF and IL-1□ were obtained from the National Institute for Biological Standards and Controls (NIBSC, Potters Bar, UK). Rat-recombinant LT-□ and mouse-recombinant TNF was purchased from R&D Systems (Abingdon, UK). The cytokines were dissolved in endotoxin-free saline (vehicle). The cytokines contained a maximum of 100 IU endotoxin/mg cytokine (corresponding to 10 ppm by weight), which, in view of previous studies, was considered negligible in the context of these experiments.

12-week-old male Wistar rats or 8-week-old NMRI mice were used for the injection of the recombinant cytokines (Charles River, Margate, UK). Both rats and mice responded to the injection of cytokines in an identical manner. In each experiment, at least three animals were used per group. All surgical procedures were performed under an operating microscope (Wild M650, Leica, Milton Keynes, UK). Stereotaxic surgery was performed as described previously (38). Briefly, anaesthetised rats were held in a stereotaxic frame. A small hole was drilled in the skull and 1 μg of TNF or LT-α, or 1 ng of IL-1β, or saline in a volume of 1 μl for rats or 0.5 μl for mice was microinjected into the striatum (an area of brain parenchyma containing both grey and white matter) with a glass capillary needle (tip<50 μm).

1.4 Tissue Collection

After appropriate survival times, animals were deeply anaesthetised with sodium pentobarbitone. Trans-cardiac perfusions were carried out using heparinised saline. Tissue was removed and either frozen in liquid nitrogen or embedded in Tissue Tek and frozen for histology.

1.5 Identification of Leukocytes and Platelets

Frozen, 10 μm-thick serial coronal sections were cut from tissue blocks. Using immunohistochemistry, neutrophils were identified using the anti-neutrophil serum HB199 (39), activated microglia cells and recruited monocytes were identified using the ED-1, and antibody recognising a lysosomal membrane marker on myeloid cells (Serotec, Oxford, UK), and total recruited leukocytes were identified using leukocyte common antigen marker with a combination of the antibodies OX1/OX30 (Serotec, Oxford, UK; Cedarlane Laboratories Ltd., Ontario, Canada). In the brain, ED-1-positive cells were subdivided into ‘parenchymal’ and ‘vessel-associated’ cells. Parenchymal cells were defined as those that were present on the ablumenal surface of the vessel and within the parenchyma whereas vessel-associated cells were defined as those cells adherent to the lumenal surface of the vasculature. The numbers of positive cells present in the brain were quantitated. For each tissue section, 4 representative fields were chosen and the average number of positive cells was calculated and expressed as number of cells per mm².

The p55 mouse anti-rat platelet GPIIa monoclonal antibody was a kind gift from Kirin Brewery Co Ltd, Takasaki, Japan. The numbers of platelet positive elements present in the brain were quantitated. For each tissue section, 6 representative fields were chosen and the average number of positive elements was calculated and expressed as number of discrete elements per mm².

For the platelet-detection in mice used for in vivo-MRI, mouse platelets were detected using rat anti-mouse glycoprotein IIb (CD41) polyclonal antibody (Clone MWReg30, GeneTex, San Antonio Tex., USA) in a dilution of 1:25 overnight at 4° C. Primary antibody was detected using a rabbit anti-rat biotinylated secondary antibody (Vectastain ABC-AP Kit, Vector, Grünberg, Germany) and an alkaline phosphate reaction (Alkaline Phosphatase Substrate Kit II, Vector, Grünberg, Germany).

Cresyl-violet-stained brain sections were examined for the presence of MPIO. Digital light microscopy (LM) images of histological sections were captured with a Cool Snap Pro colour video camera (Media Cybernetics, Silver Spring, Md.) mounted on a light microscope (Leica DM R).

1.6 Double/Triple-Labelling Immunohistochemistry/Immunofluorescence.

Frozen 10 μm coronal sections were fixed in ethanol. A rabbit polyclonal antibody to the glucose transporter (GLUT-1) was used to identify the vessel surfaces of the brain. The glucose transporter has previously been established as present on both the lumenal and ablumenal sides of all vessels within the brain (22). GLUT-1 antibody was a gift from Dr Ian Simpson, Penn State College of Medicine, Hershey, Pa., USA. GLUT-1 was detected using standard ABC procedures as described above and revealed with DAB (brown precipitate). ED-1 was subsequently identified using immunohistochemistry and revealed with VIP (blue precipitate) as described above. For double-labelling immunofluorescence, GLUT-1 was revealed with chicken anti-rabbit Alexoflor-636 (Molecular Probes, Leiden, The Netherlands) and ED-1 was identified with an anti-ED-1 directly labelled to FITC (Serotec, Oxford, UK) according to manufacturer's instructions. Sections were analysed by laser scanning confocal microscopy. Double stained images presented are all 3-dimensional reconstructions where AF-636 is presented as blue labelling and FITC as red labelling. Triple-stained images presented are all 3-dimensional reconstructions where AF-636 (GLUT-1) is presented as blue labelling and FITC (ED-1) as green labelling and RITC (platelets) as red.

1.7 In Vivo Magnetic Resonance Imaging of CM Mice

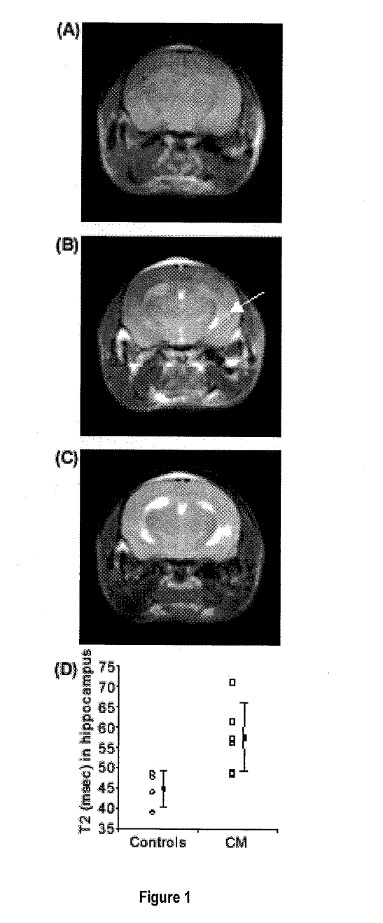

MRI data were acquired using a 7-Tesla horizontal bore magnet with a Varian Inova spectrometer (Varian, Palo Alto, Calif.). Animals (n=3 per group) were imaged at days 6 and 7 post-inoculation. An additional animal was imaged at day 5. Animals were anaesthetised with 0.5-1.5% isoflurane in 70% N₂O:30% O₂ and positioned in an Alderman-Grant resonator. Heart rate was monitored by ECG, which was maintained at approximately 500-540 beats per minute (bpm) in all animals, and body temperature was mainted at 37° C. with a MRI-compatible homeothermic blanket and probe. T₂ maps (TE=0.02, 0.04 and 0.06 sec) were acquired and regions of interest (hippocampus, cortex and striatum) were selected on the slice at the same position as those depicted in FIGS. 1A and C. T₁-weighted images (TR=0.5 sec, TE=0.02 sec) were acquired pre- and 10 min post-gadolinium DTPA (Gd) injection to assess blood-brain barrier (BBB) breakdown. A T₂*-weighted 3D gradient-echo dataset was acquired; flip angle 35°, TR=15 ms, TE=7 ms, field of view 22.5×11.2×31.6 mm, matrix size 192×96×360, 6 averages, total acquisition time ˜30 min. The mid-point of the acquisition was 1.8±0.4 h after MPIO injection (LIBS-MPIO or control-MPIO; n=3 per group). Data were zero-filled to 256×128×360 and reconstructed off-line, with a final isotropic resolution of 88 μm³.

1.8 In Vivo Magnetic Resonance Imaging of Cytokine-Injected Mice

Mice were injected via a tail vein with the LIBS-MPIO or control-MPIO contrast agent (4×10⁸ beads; 4.5 mg iron/kg body weight; n=3 per group) 11.5±3.3 h after intracerebral injection of either vehicle (saline) or recombinant cytokine (TNF/IL-1β). To examine the temporal relationship between the MRI MPIO signal and the number of platelets bound to the cerebral vasculature we also injected, via a tail vein, the LIBS-MPIO contrast agent 5.1±0.1 h or 24.2±0.1 h after the intracerebral injection of TNF.

Furthermore, to establish whether serial imaging would be possible with the LIBS-MPIO contrast agent, MRI measurements were repeated 10 hours after LIBS-MPIO contrast agent injection in mice using the protocol described above. For this purpose, animals were used which had had a TNF injection 6 h prior the initial MRI, as it was desired to demonstrate that LIBS-MPIO binding was definitely finished by the timepoint of the second scan in spite of the peaking platelet number detected 12 hours after TNF-injection.

Following MPIO injection, animals were placed in an Alderman-Grant resonator and positioned in the magnet. During MRI, anaesthesia was maintained with 1.7-2.5% isoflurane in 70% N₂O:30% O₂, ECG was monitored throughout and body temperature was maintained at ˜37° C. with a circulating warm water system. A T₂*-weighted 3D gradient-echo dataset was acquired; flip angle 35°, TR=50 ms, TE=5 ms, field of view 22.5×22.5×31.6 mm, matrix size 192×192×360, 2 averages, total acquisition time ˜1 hour. The mid-point of the acquisition was 1.8±0.2 h after MPIO injection. Data were zero-filled to 256×256×360 and reconstructed off-line, with a final isotropic resolution of 88 μm³. All in vivo procedures were approved by the United Kingdom Home Office.

1.9 MRI Data Analysis

In each MR image the brain was masked manually to exclude extra-cerebral structures. Quantitative analysis was undertaken in 41 contiguous slices per brain, spanning a depth of 3.6 mm from the dorsal hippocampus ventrally. Areas of low signal were segmented. To control for minor variations in absolute signal intensity between individual scans, low signal areas were calibrated on 10 evenly spaced slices per brain. The median signal intensity value was then applied to signal intensity histogram-based fully automated batch analysis of the entire 41 slice sequence. In this way, masks were generated corresponding to areas that were both within the brain and of defined low signal intensity. Voxel volumes were summated and expressed as raw volumes in μm³ with no surface rendering or smoothing effects. Segmentation and volumetric quantification were undertaken using ImagePro Plus software (version 4.5.1, Media Cybernetic, Silver Spring Md.) by an operator blinded to the origin of all data.

1.10 Statistical Methods

The data were presented as mean±standard error of the mean at each time point. Where statistical analysis was employed, data were analysed by t-tests. Results were considered significant when p<0.05.

### Example 2

### Imaging of CM with Conventional MRI

Conventional MRI was performed throughout the development of disease in mice infected with the CM parasite. No abnormalities were detected until day 7, when breakdown of the BBB was evident as hyperintense areas on T₁-weighted images obtained after injection of Gadopentetic acid (Gd-DTPA), which were not present prior to Gd-DTPA injection (FIG. 1A, B). Discrete hyperintensities were also present on T₂-weighted images (FIG. 1C), which coincided with the Gd-DTPA-enhancing lesions. The evaluation of T₂ maps revealed a significant increase in T₂ within the hippocampus (FIG. 1D). However, by day 7 the mice were moribund, and MRI at this time provides little additional information on the pathogenic process. Before day 7, no overt clinical signs were evident. Thus our studies employing conventional MRI techniques failed to reveal the presence of CNS pathology before the appearance of overt clinical signs

### Example 3

### Imaging of CM Using a Platelet-Specific Contrast Agent

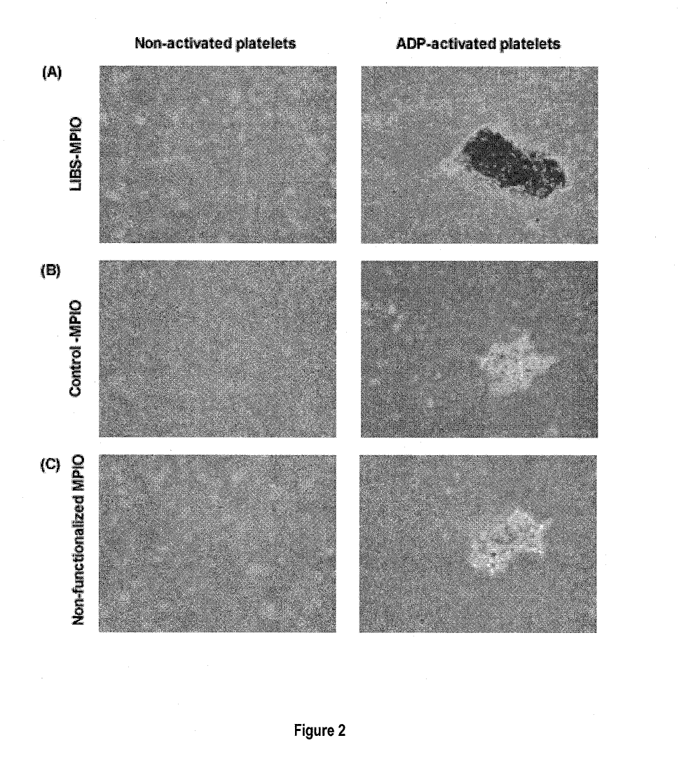

Murine and human CM is associated with the adherence of platelets to an intact brain endothelium (20, 21). The aim of this study was to determine whether we could distinguish between CM and control mice at a time when no overt disease was present using a novel contrast agent that recognises activated platelets. The in vitro experiments revealed that the functionalized MPIOs, which recognize the ligand induced binding sites of GPIIb/IIIa receptors (LIBS-MPIO), bind to ADP-activated platelets alone. No significant binding was observed with control-MPIO or non-functionalized MPIOs (FIG. 2).

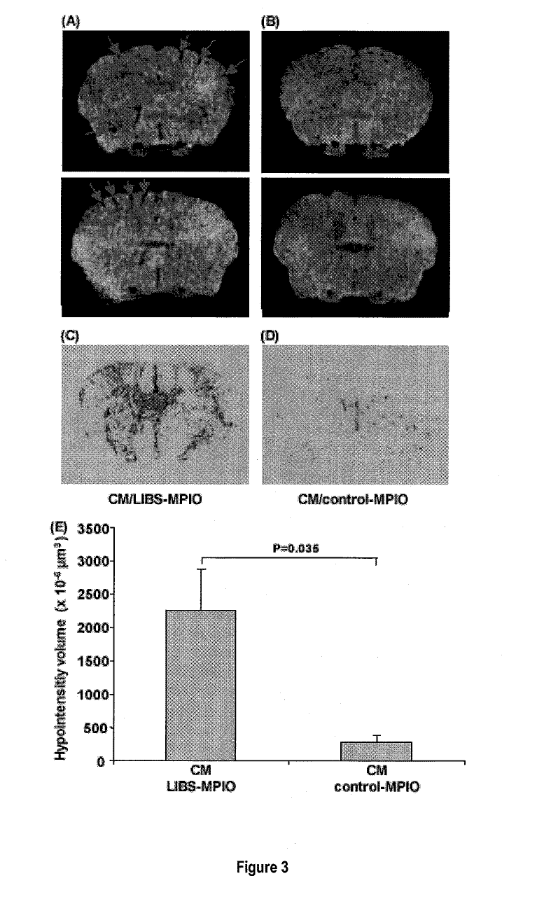

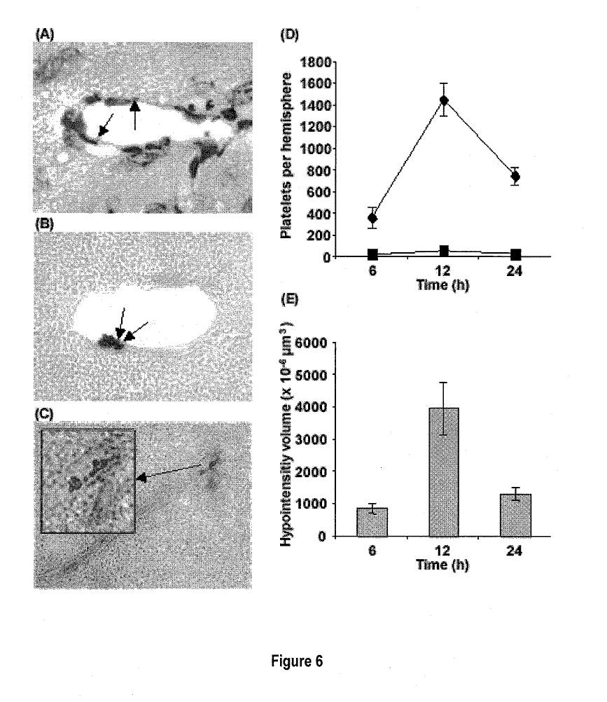

When MRI was performed at day 5 after Plasmodium berghei ANKA infection, some contrast enhancement, which appear as focal hypointensities, was observed after LIBS-MPIO injection. However, after injection of LIBS-MPIO in CM-infected mice at day 6, MPIO-associated MRI contrast was evident in and around cortical vessels, using a T₂*-weighted 3D gradient-echo sequence, delineating areas of LIBS-MPIO binding as demonstrated in two representative slices at different levels of the same brain (red arrows, FIG. 3A). Conversely, CM-infected animals injected with control-MPIO exhibited no negative contrast in corresponding areas (FIG. 3B). Using a 3D reconstruction of the original MRI data stack, it can be seen that binding of LIBS-MPIO is enhanced in cortical regions of the brain (FIG. 3C), whilst injection of control-MPIO does not give rise to specific binding (FIG. 3D). Using volumetric quantification, a significant increase in the extent of signal voids per volume was confirmed for the LIBS-MPIO-injected animals compared to control-MPIO injected animals as depicted in FIG. 3E (2261+/−623 vs. 282+/−101; P=0.035). Furthermore, histological evaluation of LIBS-injected CM animals revealed the presence of MPIO bound to aggregated endovascular platelets (FIG. 6C).

These data confirm that there is significant binding of LIBS-MPIO to areas of CM pathology, and demonstrate that the use of this novel targeted contrast agent enables detection of pathology before the onset of overt clinical signs.

### Example 4

### In Vivo MRI for Platelet Detection after TNF and IL-1β Injection

To further investigate the mechanisms underlying cerebrovascular platelet aggregation in cerebral malaria, MRI was used in conjunction with the LIBS-MPIO compound to examine the spatial distribution of platelet aggregation in vivo following stereotactic injection of either TNF or IL-1□ into the brains of normal mice.

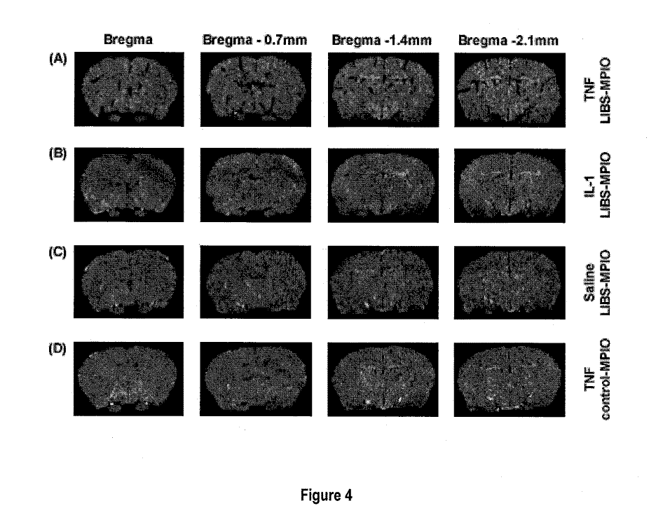

In the TNF-injected animals using the LIBS-MPIO compound, negative MRI contrast was observed bilaterally throughout the anterior portion of the forebrain (FIG. 4A). Conversely, animals injected intracerebrally with IL-1β (FIG. 4B) or saline (FIG. 4C) showed no areas of MPIO-binding (negative contrast) following injection of LIBS-MPIO. Non-specific binding of LIBS-MPIO was excluded using control-MPIO in TNF-injected mice (FIG. 4D). As with mice injected with IL-1β or saline, these animals showed no alteration in signal intensity.

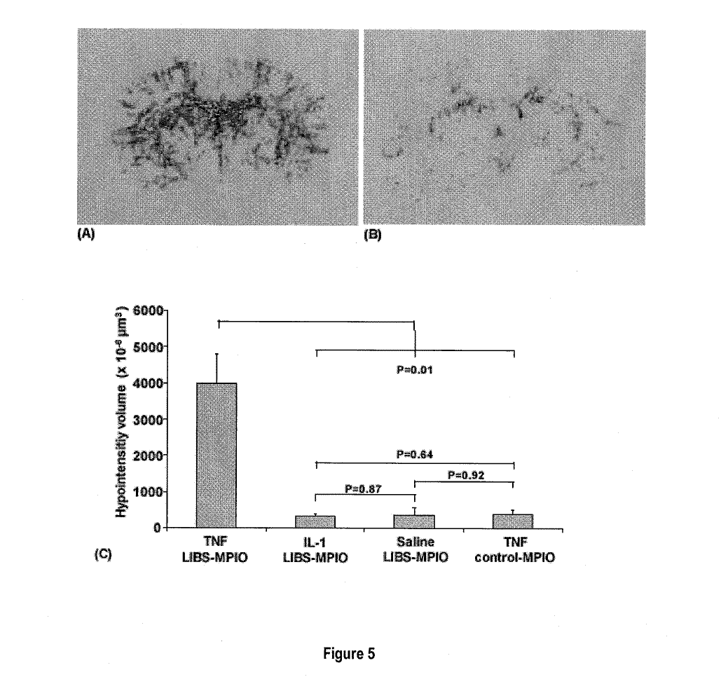

Using a 3D reconstruction of the original MRI data stack, it can be seen that binding of LIBS-MPIO is enhanced in both cortical and striatal regions of the brain following TNF injection into the brain parenchyma (FIG. 5A), whilst no specific binding was evident following control-MPIO injection (FIG. 5B). As expected, 12 h after TNF injection a similar binding pattern is found in both hemispheres, indicating bilateral aggregation, with slightly enhanced signal intensity changes on the side of injection. Volumetric quantification confirmed significantly (P<0.05) greater LIBS-MPIO binding in TNF-injected animals compared to both IL-1β and saline injected animals, as well as TNF-injected animals receiving the control-MPIO agent (FIG. 5C). These in vivo data indicate a as role for TNF in platelet aggregation in cerebral vessels.

Immunohistochemically, MPIO binding was apparent in TNF-injected animals receiving the LIBS-MPIO (FIG. 6). On cresyl-violet-stained paraffin-embedded sections, binding of MPIOs to areas near the vascular wall was confirmed (FIG. 6A). Furthermore, co-staining with CD41 (GPIIb subunit), confirmed the binding of LIBS-MPIO was specific to areas of wall-adherent platelets (FIG. 6B). Attached to the red-stained thrombus area, two MPIOs in different focus (owing to their location in different focal planes through the section) are evident. The number of platelets bound to the brain endothelium after the microinjection of TNF is highest at 12 hours both in rats and in mice. To examine the relationship between the hypointensitiy volume and the number of platelets adherent in the vasculature the MRI signal was compared with platelet immunohistochemistry at 6 h, 12 h, and 24 h after the microinjection of TNF in mice. Maximum binding of the contrast agent to the microvasculature is also maximal at 12 h as detected by MRI, and there is a correlation between the number of platelets present in the brain vasculature and the MPIO-dependent signal loss. This suggests that increased platelet load over time is directly related to increased LIBS-MPIO induced signal void. In animals injected with LIBS-MPIO at 5.1 h after TNF and imaged both at 6.5 h and at 16 h the initial signal from the LIBS-MPIO present at one hour after agent injection was absent 10 hours later. This effect was clear in spite of the fact that the second scan was performed at a timepoint around peak platelet adhesion in the brain.

### Example 5

### Cytokines and Platelet Aggregation

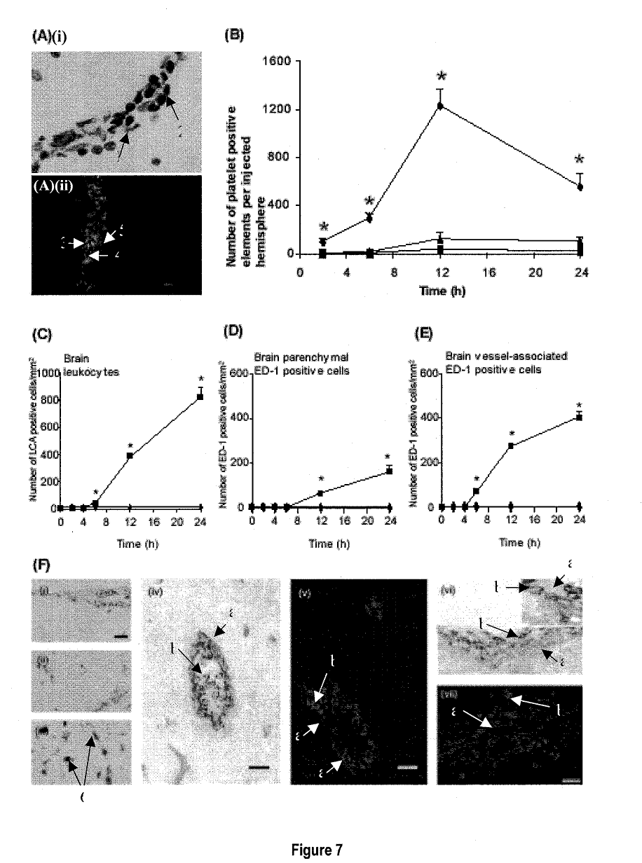

To establish whether the early expression of cytokines within the brain parenchyma is responsible for platelet adhesion, TNF, LT-α or IL-1β was injected directly into the brain parenchyma. The role of these cytokines in platelet adherence was hitherto unknown. GPIIb-positive platelets become adherent to the lumenal portion of the vasculature from two hours after microinjection of TNF into the rat brain parenchyma (FIGS. 7A and 7B). The number of platelet-positive elements peaked at 12 hours, but was still significantly increased 24 h after injection of TNF. This effect was also observed in mouse brain parenchyma, where platelet adhesion at the 12 h time point reached the maximum compared to the 6 h or 24 h timepoints (FIG. 6D). In contrast, injection of IL-1β, LT-α or vehicle did not increase the number of platelet positive elements (FIG. 7B). Thus it would appear that platelet binding to the brain endothelium is cytokine-specific, and that TNF is likely to be the principal mediator of endothelial platelet binding in CM. Interestingly, leukocytes, identified by the leukocyte common antigen, were recruited to the brain parenchyma or became adherent to the lumenal portion of the vasculature over the 24 h period following injection of TNF into the striatum, but after the appearance of platelets in the microvasculature (FIG. 7C). The recruited leukocytes were principally ED-1-positive cells, and no neutrophils were observed (results not shown). The recruited ED-1-positive cells could be distinguished as two separate populations: those which had diapedesed into the parenchyma (‘parenchymal ED-1-positive cells’) (FIG. 7D) and those which were associated with the lumenal portion of the brain vasculature that appeared to be unable to diapedese into the parenchyma (‘vessel-associated ED-1-positive cells’) (FIG. 7E). The number of ED-1-positive cells detected in the brain parenchyma increased from 12 h post-injection (p<0.05) and peaked at 24 h (p<0.05), compared to vehicle-injected controls. A larger number of vessel-associated ED-1 positive cells was observed after 6 h (p<0.05), again increasing over time to peak at 24 h (p<0.05) as compared to vehicle-injected controls. Representative histology pictures demonstrate the lack of cellular recruitment in the meninges (FIG. 7F i) and parenchyma (FIG. 7F ii) of vehicle-injected controls and the presence of ED-1 positive cells in the parenchyma after TNF □microinjection into the striatum (FIG. 7F iii). The ED-1-positive cells adherent to the vessel lumen were visualised using double-labelling immunohistochemistry (FIG. 7F iv): vessels were located using an antibody to the glucose transporter (GLUT-1), a marker whose expression has been established on both the lumenal and ablumenal portions of the brain vasculature (22), together with an antibody to ED-1. These findings were confirmed using immunofluorescence and laser-scanning confocal microscopy (FIG. 7F v). Interestingly, the GLUT-1 confocal microscopy revealed that the platelets were binding to intact endothelium which was unexpected.

In the meninges, recruited ED-1-positive mononuclear cells were observed as early as 2 h after intrastriatal TNF injection, and increased dramatically over the 24 h period (results not shown). The movement of ED-1-positive mononuclear cells across the meningeal vasculature appeared unrestricted, as large numbers of ED-1-positive cells were observed ablumenally (FIG. 7F vi). These findings were supported by immunofluorescence and confocal microscopy (FIG. 7F vii). Although not observed in the brain parenchyma, neutrophils were found in the meninges. These findings were also true of the choroid plexus, where a similar acute inflammatory response was to displayed after 2 h increasing to 24 h (results not shown).

### Example 6

### Early Detection of Experimental Autoimmune Encephalomyelitis (EAE) in Mice Using GPIIb/IIIa-Targeted Super Paramagnetic Iron Oxide (SPIO) Particles in Magnetic Resonance Imaging (MRI)

Experimental Autoimmune Encephalomyelitis (EAE) is an animal model of multiple sclerosis (MS). It is an acute, acquired, inflammatory and demyelinating autoimmune disease. In this animal model, animals are injected with the whole or parts of various proteins that make up myelin, which is the insulating sheath that surrounds nerve cells (neurons). These proteins induce an autoimmune response in the animals. That is, the animal's immune system mounts an attack on its own myelin as a result of exposure to the injection. The animals then develop a disease process that closely resembles MS in humans.

To induce EAE in mice in the present study, mice were immunized subcutaneously in each hind flank with 100 μg of a peptide derived from the sequence of a myelin component called myelin oligodendrocyte glycoprotein (MOG, a 35-55 peptide) emulsified in Freund's complete adjuvant, with 4 mg/ml of Mycobacterium tuberculosis added (100 μl in total in each flank). Immediately afterwards, mice were injected in the lateral tail vein with 300 μl of phosphate buffered saline containing 350 ng of Pertussis toxin. The Pertussis toxin injection was repeated 48 hrs later. Mice were monitored for signs of weight loss and clinical symptoms. Sham control mice were also injected as above, except for the omission of MOG 35-55 peptide.

After 7, 10, 14 and 17 days, mice underwent MRI scans under anesthesia. The animals were placed in an anesthetic chamber and breathed 5% Isoflurane in medical grade oxygen. Once mice were anaesthetized (about 3-5 minutes), they were transferred to a purpose-built Perspex holder and a nose cone was placed over the front of the head. The Isoflurane concentration was then reduced to 1 to 1.5% to maintain anesthesia via the nose cone for the remainder of the imaging experiment. The Perspex holder and the mouse were then placed into the magnet and the imaging process commenced. Respiratory rate was continuously monitored throughout the experiment using a probe placed under the animal's body.

T2 (TR 5000 ms, TE 53.6 ms) weighted images were acquired in single-slice mode (15 slices axial and 8 slices coronal) with a FOV (field of view) of 2 cm×2 cm, a slice thickness of 1.0 mm with matrix of 256×256, average 12. A small body coil was used for transmission. Pre-contrast images were acquired. Targeted nanoparticles (4×10⁸ SPIO in saline) were infused. Control mice had non-targeted SPIOs infused. Final MR images were taken after injection of contrast agent.

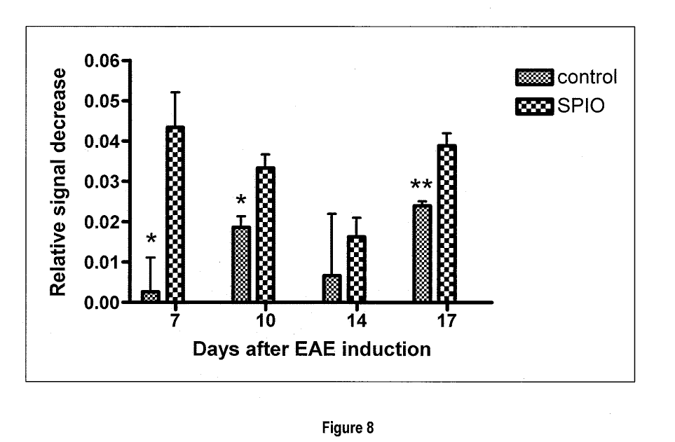

As shown in the FIG. 8, strong differences could be shown in MRI signal decrease as early as day 7 between animals injected with targeted SPIOs compared to animals injected with non-targeted SPIOs. Animals at day 7 had a clinical score of 0 (no symptoms).

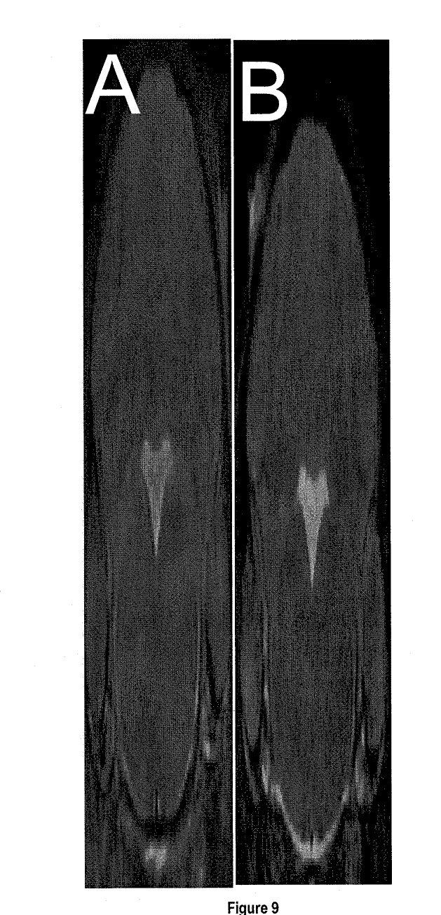

As shown in FIG. 9, binding of targeted SPIOs could be shown in the cerebellum as a diffuse staining of the vasculature.

### REFERENCES

1. Marsh, K., and Snow, R. W. 1999. Malaria transmission and morbidity. Parassitologia 41:241-246.
2. Wassmer, S. C., Combes, V., Candal, F. J., Juhan-Vague, I., and Grau, G. E. 2006. Platelets potentiate brain endothelial alterations induced by Plasmodium falciparum. Infect Immun 74:645-653.
3. Grau, G. E., Tacchini-Cottier, F., Vesin, C., Milon, G., Lou, J. N., Piguet, P. F., and Juillard, P. 1993. TNF-induced microvascular pathology: active role for platelets and importance of the LFA-1/ICAM-1 interaction. Eur Cytokine Netw 4:415-419.
4. Grau, G. E., and Lou, J. 1993. TNF in vascular pathology: the importance of platelet-endothelium interactions. Res Immunol 144:355-363.
5. Combes, V., Rosenkranz, A. R., Redard, M., Pizzolato, G., Lepidi, H., Vestweber, D., Mayadas, T. N., and Grau, G. E. 2004. Pathogenic role of P-selectin in experimental cerebral malaria: importance of the endothelial compartment. Am J Pathol 164:781-786.
6. Grau, G. E., Mackenzie, C. D., Carr, R. A., Redard, M., Pizzolato, G., Allasia, C., Cataldo, C., Taylor, T. E., and Molyneux, M. E. 2003. Platelet accumulation in brain microvessels in fatal pediatric cerebral malaria. J Infect Dis 187:461-466.
7. Lou, J., Donati, Y. R., Juillard, P., Giroud, C., Vesin, C., Mili, N., and Grau, G. E. 1997. Platelets play an important role in TNF-induced microvascular endothelial cell pathology. Am J Pathol 151:1397-1405.
8. Grau, G. E., and Lou, J. N. 1995. Experimental cerebral malaria: possible new mechanisms in the TNF-induced microvascular pathology. Soz Praventivmed 40:50-57.
9. Sibson, N. R., Blamire, A. M., Bernades-Silva, M., Laurent, S., Boutry, S., Muller, R. N., Styles, P., and Anthony, D. C. 2004. MRI detection of early endothelial activation in brain inflammation. Magn Reson Med 51:248-252.
10. Hunt, N. H., and Grau, G. E. 2003. Cytokines: accelerators and brakes in the pathogenesis of cerebral malaria. Trends Immunol 24:491-499.
11. Spuentrup, E., Buecker, A., Katoh, M., Wiethoff, A. J., Parsons, E. C., Jr., Botnar, R. M., Weisskoff, R. M., Graham, P. B., Manning, W. J., and Gunther, R. W. 2005. Molecular magnetic resonance imaging of coronary thrombosis and pulmonary emboli with a novel fibrin-targeted contrast agent. Circulation 111:1377-1382.
12. Nahrendorf, M., Jaffer, F. A., Kelly, K. A., Sosnovik, D. E., Aikawa, E., Libby, P., and Weissleder, R. 2006. Noninvasive vascular cell adhesion molecule-1 imaging identifies inflammatory activation of cells in atherosclerosis. Circulation 114:1504-1511.
13. Shapiro, E. M., Skrtic, S., and Koretsky, A. P. 2005. Sizing it up: cellular MRI using micron-sized iron oxide particles. Magn Reson Med 53:329-338.
14. Shapiro, E. M., Skrtic, S., Sharer, K., Hill, J. M., Dunbar, C. E., and Koretsky, A. P. 2004. MRI detection of single particles for cellular imaging. Proc Natl Acad Sci USA 101:10901-10906.
15. McAteer, M. A., Sibson, N. R., von Zur Muhlen, C., Schneider, J. E., Lowe, A. S., Warrick, N., Channon, K. M., Anthony, D. C., and Choudhury, R. P. 2007. In vivo magnetic resonance imaging of acute brain inflammation using microparticles of iron oxide. Nat Med 13:1253-1258.
16. Schwarz, M., Meade, G., Stoll, P., Ylanne, J., Bassler, N., Chen, Y. C., Hagemeyer, C. E., Ahrens, I., Moran, N., Kenny, D., et al. 2006. Conformation-specific blockade of the integrin GPIIb/IIIa: a novel antiplatelet strategy that selectively targets activated platelets. Circ Res 99:25-33.
17. Schwarz, M., Rottgen, P., Takada, Y., Le Gall, F., Knackmuss, S., Bassler, N., Buttner, C., Little, M., Bode, C., and Peter, K. 2004. Single-chain antibodies for the conformation-specific blockade of activated platelet integrin alphallbbeta3 designed by subtractive selection from naive human phage libraries. Faseb J 18:1704-1706.
18. Stoll P, B. N., Hagemeyer C, Eisenhardt S, Chih C, Schmidt R, Schwarz M, Ahrens I, Katagiri Y, Pannen B, Bode C, Peter K. 2007. Targeting ligand-induced binding sites on GPIIb/IIIa via single-chain antibody allows effective anticoagulation without bleeding time prolongation. ATVB in press.
19. von zur Mühlen, C., Peter, K., Ali, Z., Schneider, J., McAteer, M. A., Channon, K. M., Bode, C., and Choudhury, R. P. 2007. Magnetic resonance imaging of platelets on wire-injured mouse femoral arteries using activation-specific anti-GP IIb/IIIa single chain antibodies conjugated to microparticles of iron oxide JACC abstract suppl 49:108.
20. Sun, G., Chang, W. L., Li, J., Berney, S. M., Kimpel, D., and van der Heyde, H. C. 2003. Inhibition of platelet adherence to brain microvasculature protects against severe Plasmodium berghei malaria. Infect Immun 71:6553-6561.
21. van der Heyde, H. C., Nolan, J., Combes, V., Gramaglia, I., and Grau, G. E. 2006. A unified hypothesis for the genesis of cerebral malaria: sequestration, inflammation and hemostasis leading to microcirculatory dysfunction. Trends Parasitol 22:503-508.
22. Guerin, C., Laterra, J., Hruban, R. H., Brem, H., Drewes, L. R., and Goldstein, G. W. 1990. The glucose transporter and blood-brain barrier of human brain tumors. Ann Neurol 28:758-765.
23. Winter, P. M., Caruthers, S. D., Yu, X., Song, S. K., Chen, J., Miller, B., Bulte, J. W., Robertson, J. D., Gaffney, P. J., Wickline, S. A., et al. 2003. Improved molecular imaging contrast agent for detection of human thrombus. Magn Reson Med 50:411-416.
24. Lipinski, M. J., Amirbekian, V., Frias, J. C., Aguinaldo, J. G., Mani, V., Briley-Saebo, K. C., Fuster, V., Failon, J. T., Fisher, E. A., and Fayad, Z. A. 2006. MRI to detect atherosclerosis with gadolinium-containing immunomicelles targeting the macrophage scavenger receptor. Magn Reson Med 56:601-610.
25. Runge, V. M., Schoerner, W., Niendorf, H. P., Laniado, M., Koehler, D., Claussen, C., Felix, R., and James, A. E., Jr. 1985. Initial clinical evaluation of gadolinium DTPA for contrast-enhanced magnetic resonance imaging. Magn Reson Imaging 3:27-35.
26. Sipkins, D. A., Gijbels, K., Tropper, F. D., Bednarski, M., Li, K. C., and Steinman, L. 2000. ICAM-1 expression in autoimmune encephalitis visualized using magnetic resonance imaging. J Neuroimmunol 104:1-9.
27. Shapiro, E. M., Sharer, K., Skrtic, S., and Koretsky, A. P. 2006. In vivo detection of single cells by MRI. Magn Reson Med 55:242-249.
28. Sibson, N. R., Blamire, A. M., Perry, V. H., Gauldie, J., Styles, P., and Anthony, D. C. 2002. TNF-alpha reduces cerebral blood volume and disrupts tissue homeostasis via an endothelin- and TNFR2-dependent pathway. Brain 125:2446-2459.
29. McAteer, M. A., Schneider, J. E., Ali, Z. A., Warrick, N., Bursill, C. A., von Zur Muhlen, C., Greaves, D. R., Neubauer, S., Channon, K. M., and Choudhury, R. P. 2007. Magnetic Resonance Imaging of Endothelial Adhesion Molecules in Mouse Atherosclerosis Using Dual-Targeted Microparticles of Iron Oxide. Arterioscier Thromb Vasc Biol. in press 2007
30. Bell, M. D., and Perry, V. H. 1995. Adhesion molecule expression on murine cerebral endothelium following the injection of a proinflammagen or during acute neuronal degeneration. J Neurocytol 24:695-710.
31. McHale, J. F., Harari, O. A., Marshall, D., and Haskard, D. O. 1999. Vascular endothelial cell expression of ICAM-1 and VCAM-1 at the onset of eliciting contact hypersensitivity in mice: evidence for a dominant role of TNF-alpha. J Immunol 162:1648-1655.
32. Proescholdt, M. G., Chakravarty, S., Foster, J. A., Foti, S. B., Briley, E. M., and Herkenham, M. 2002. Intracerebroventricular but not intravenous interleukin-1beta induces widespread vascular-mediated leukocyte infiltration and immune signal mRNA expression followed by brain-wide glial activation. Neuroscience 112:731-749.
33. Blamire, A. M., Anthony, D. C., Rajagopalan, B., Sibson, N. R., Perry, V. H., and Styles, P. 2000. Interleukin-1beta-induced changes in blood-brain barrier permeability, apparent diffusion coefficient, and cerebral blood volume in the rat brain: a magnetic resonance study. J Neurosci 20:8153-8159.
34. Schwarz, M., Katagiri, Y., Kotani, M., Bassler, N., Loeffler, C., Bode, C., and Peter, K. 2004. Reversibility versus persistence of GPIIb/IIIa blocker-induced conformational change of GPIIb/IIIa (alphallbbeta3, CD41/CD61). J Pharmacol Exp Ther 308:1002-1011.
35. Wilcockson, D. C., Campbell, S. J., Anthony, D. C., and Perry, V. H. 2002. The systemic and local acute phase response following acute brain injury. J Cereb Blood Flow Metab 22:318-326.
36. Blond, D., Campbell, S. J., Butchart, A. G., Perry, V. H., and Anthony, D. C. 2002. Differential induction of interleukin-1 beta and tumour necrosis factor-alpha may account for specific patterns of leukocyte recruitment in the brain. Brain Res 958:89-99.
37. Andersson, P. B., Perry, V. H., and Gordon, S. 1992. The acute inflammatory response to lipopolysaccharide in CNS parenchyma differs from that in other body tissues. Neuroscience 48:169-186.
38. Matyszak, M. K., and Perry, V. H. 1995. Demyelination in the central nervous system following a delayed-type hypersensitivity response to bacillus Calmette-Guerin. Neuroscience 64:967-977.
39. Anthony, D., Dempster, R., Fearn, S., Clements, J., Wells, G., Perry, V. H., and Walker, K. 1998. CXC chemokines generate age-related increases in neutrophil-mediated brain inflammation and blood-brain barrier breakdown. Curr Biol 8:923-926.

What is claimed is:

1. A method for diagnosing, predicting or monitoring a disease in a subject, wherein the method comprises administering to the subject a compound comprising:
(a) a binding element capable of specifically binding to an activated platelet; and
(b) an imaging agent
wherein binding of the compound to an activated platelet is indicative of the disease.

2. The method according to claim 1, wherein the diagnosing, predicting or monitoring comprises magnetic resonance imaging.

3. The method according to claim 1, wherein the disease is selected from the group comprising stroke, thrombosis, cardiovascular disease, inflammatory disease, autoimmune disease, immunoinflammatory disease, allergic disease, predispositions thereto, infectious disease and cancer.

4. The method according to claim 1, wherein the monitoring comprises monitoring responses to therapy for stroke, thrombosis, cardiovascular disease, inflammatory disease, autoimmune disease, immunoinflammatory disease, allergic disease, predispositions thereto, infectious disease and cancer.

5. A method for treating a disease in a subject, wherein the method comprises administering to the subject a compound comprising:
(a) a binding element capable of specifically binding to an activated platelet; and
(b) an agent that inhibits TNF
wherein binding of the compound to an activated platelet facilitates inhibition of TNF signaling by the agent.

6. A method of non-invasively detecting vascular platelet aggregation in a subject comprising:
(a) administering to the subject a composition comprising a binding element that specifically binds activated platelets conjugated to an imaging agent, wherein the composition has substantially no effect upon platelet aggregation;
(b) allowing the binding element to bind to any activated platelets present in the subject; and
(c) imaging the imaging agent, wherein an image signals the detection of vascular platelet aggregation.

7. The method according to claim 6, further comprising the steps of:
(d) allowing for clearance of the imaging agent from the subject sufficient to eliminate or reduce its detection; and
(e) repeating steps (a) through (c).
